(19) United States
(12) Patent Application Publication
(10) Pub. No.: US 2025/0025550 A1
(43) Pub. Date: Jan. 23, 2025

(54) ADJUVANT COMPOUNDS AND FORMULATIONS

(71) Applicant: ADJUVANCE TECHNOLOGIES, INC., Lincoln, NE (US)

(72) Inventors: Jared William WENGER, Lincoln, NE (US); Harsh Vardhan CHAUHAN, Omaha, NE (US)

(21) Appl. No.: 18/708,417

(22) PCT Filed: Nov. 8, 2022
(86) PCT No.: PCT/US2022/049255

§ 371 Date: May 8, 2024

Related U.S. Application Data
(60) Provisional application No. 63/277,001, filed on Nov. 8, 2021.
(60) Provisional application No. 63/351,057, filed on Jun. 10, 2022.

(51) Int. Cl. A61K 39/39; A61K 39/00; A61K 39/25

(52) U.S. Cl. CPC A61K 39/39; A61K 39/25; A61K 2039/55583

(57) ABSTRACT

The present application relates to triterpene glycoside saponin-derived adjuvants, syntheses thereof, and intermediates thereto. The application also provides formulations and pharmaceutical compositions comprising compounds of the present invention and methods of using said compounds, formulations, or compositions in the treatment of and immunization for infectious diseases.

### INCORPORATION BY REFERENCE OF RELATED APPLICATIONS

This application is based upon and claims priority under 35 U.S.C. § 119(e) to U.S. Provisional Application U.S. Ser. No. 63/277,001, filed Nov. 8, 2021, and to U.S. Provisional Application U.S. Ser. No. 63/351,057, filed Jun. 10, 2022, the entire contents of all of which are incorporated herein by reference in their entirety.

### FIELD OF THE INVENTION

The present application relates to triterpene glycoside saponin-derived adjuvants and pharmaceutical compositions comprising compounds of the present invention, as well as methods of making and of using the foregoing in the treatment of certain diseases.

### BACKGROUND

Vaccines against infectious diseases continue to improve public health across the world. With increased knowledge of etiologic pathogens and necessary immune responses have come increasingly defined or targeted vaccines. Hepatitis B, DTaP, HPV, pneumococcal and other widely used vaccines require use of the immunological adjuvant alum. However, alum, which was introduced over 80 years ago, is a poor adjuvant restricting the potency of some of these vaccines and requiring higher or more doses of others. A far more potent adjuvant employed more recently is the natural saponin adjuvant QS-21, used widely despite 3 major liabilities: dose limiting toxicity, poor stability, and limited availability of quality product.

Saponins are glycosidic compounds that are produced as secondary metabolites of steroids and triterpenes. They are widely distributed among plant species and in some marine invertebrates. The chemical structure of saponins imparts a wide range of pharmacological and biological activities, including some potent and efficacious immunological activity. Semi-purified saponin extracts from the bark of the South American Quillaja saponaria Molina tree (Quillaja saponins) exhibit remarkable immunoadjuvant activity. Because the Quillaja saponins are found as a mixture of at least one hundred structurally related saponin glycosides, their separation and isolation is often difficult if not prohibitive. The most active fraction of these extracts, designated QS-21, has been found to include a mixture of two principal isomeric triterpene glycoside saponins, each incorporating a quillaic acid triterpene core, flanked on either side by complex oligosaccharides and a stereochemically rich glycosylated fatty acyl chain.

The potency of QS-21 and its favorable toxicity profile in dozens of recent and ongoing vaccine clinical trials (melanoma, breast cancer, small cell lung cancer, prostate cancer, HIV-1, malaria) have established it as a promising new adjuvant for immune response potentiation and dose-sparing. However, the tolerated dose of QS-21 in cancer patients does not exceed 100-150 μg, above which significant local and systemic side effects arise. The highest practical tolerable dose in well (non-cancer) adult and child recipients is 25-50 mcg, an immunologically suboptimal dose. As a result, the clinical success of non-cancer vaccines continues to critically depend on the identification of, and access to, novel, potent adjuvants that are more tolerable.

In view of the drawbacks associated with QS-21, synthetic saponin molecules have been explored in an effort to reduce toxicity, improve stability, and increase availability. Several such saponins are described in PCT/US2009/039954, PCT/US2015/33567, PCT/US2016/67530, and PCT/US2016/60564. In particular, the saponin adjuvant TQL-1055 (Compound I-9) discussed in PCT/US2016/60564 has emerged as a leading QS-21 analogue, exhibiting reduced toxicity and improved efficacy as compared to the natural product. However, the free forms of synthetic saponin molecules, including the free acid form of TQL-1055, possess some undesirable physiochemical and/or biopharmaceutical properties, for example poor solubility in water at physiological pH, meaning the free forms of these compounds require additional formulation techniques or excipients to improve their delivery and enable widespread (e.g., commercial) pharmaceutical use.

Accordingly, the inventors of the present application invented certain stable formulations of synthetic saponin molecules, including TQL-1055, which substantially improved physicochemical and/or biopharmaceutical properties, for example poor solubility in aqueous-based solutions, such that these formulations are useful for widespread (e.g., commercial) pharmaceutical use. Such subject matter is described in U.S. Provisional Patent Application Ser. No. 63/277,001, which application is incorporated herein by reference in its entirety. Among other inventions, this application sets forth a formulation approach providing stable formulations containing saponins, such as TQL-1055, and cyclodextrins, preferably beta-cyclodextrins, and more preferably HP-β-CD (2-hydroxypropyl beta-cyclodextrin) or SBECD (sulfobutylether-β-cyclodextrin), and most preferably HP-β-CD (which may also be referred to as HPβCD).

To explore the dose-response of the foregoing stable cyclodextrin formulations, experiments were conducted using a formulation containing an antigen and TQL-1055 FA (free acid) HP-R-CD; however, higher doses of this formulation unexpectedly resulted in mortality in mice. Further follow-on studies demonstrated (1) TQL-1055 administered without HP-β-CD did not result in mortality at any dose tested, and (2) HP-β-CD administered without TQL-1055 did not result in mortality at any dose tested; and (3) certain dose levels of the combination of TQL-1055 and HP-β-CD unexpectedly resulted in murine mortality.

Accordingly, there remains a need for stable formulations of synthetic saponin molecules, including TQL-1055, which are tolerable in mammals and substantially improve physiochemical and/or biopharmaceutical properties, for example poor solubility in aqueous-based solutions, such that these formulations are useful for widespread (e.g., commercial) pharmaceutical use.

### SUMMARY

The inventors of the present application have invented certain stable formulations of synthetic saponin molecules, including TQL-1055, which contain cyclodextrins but surprisingly are tolerable in mammals and substantially improve physiochemical and/or biopharmaceutical properties, for example poor solubility in aqueous-based solutions, such that these formulations are useful for widespread (e.g., commercial) pharmaceutical use.

Thus, in certain embodiments, the present application provides stable formulations containing TQL-1055 salt and a cyclodextrin. In a preferred embodiment, the stable formulation of the present application contains TQL-1055 potassium salt and HP-R-CD. In some embodiments, the TQL-1055 potassium salt is an in situ potassium salt.

In another aspect, the present application provides stable formulations containing TQL-1055 and cyclodextrins, preferably beta-cyclodextrins, and more preferably HP-β-CD (2-hydroxypropyl beta-cyclodextrin) or SBECD (sulfobutylether-β-cyclodextrin), and most preferably HP-β-CD (which may also be referred to as HPβCD). Thus, in certain embodiments, the present application provides formulations of saponin derivatives that are tolerable in mammals and soluble and stable at physiological pH in various solvents, including e.g. water, buffers (including but not limited to phosphate-based buffers), alcohols (including but not limited to methanol, ethanol, butanol, etc.), polyols (including but not limited to glycerol, propylene glycol, polyethylene glycol, etc.), and suitable mixtures thereof, vegetable oils, such as olive oil, and injectable organic esters, such as ethyl oleate.

In addition to forms and formulations disclosed herein, the present application also discloses related methods.

In one aspect, the present application provides TQL-1055:

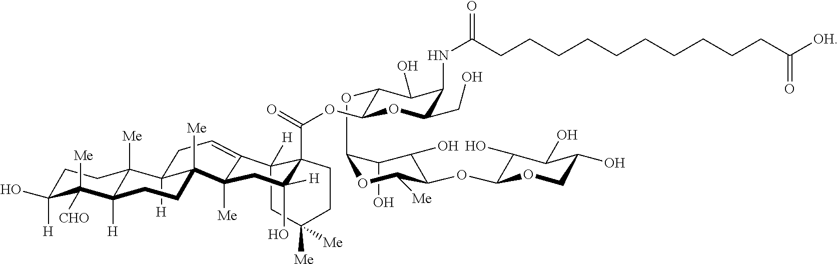

In another aspect, the present application provides compounds of Formula I:

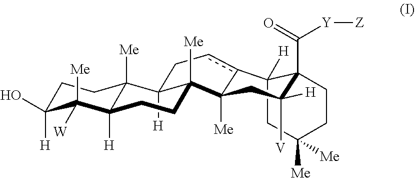

or a pharmaceutically acceptable salt thereof, wherein
[symbol] is a single or double bond;
W is —CHO;
V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

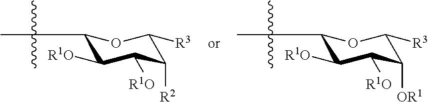

wherein each occurrence of R¹ is R^{x} or a carbohydrate domain having the structure:

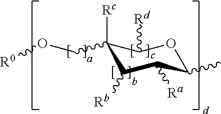

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is —T—R^{z}, —C(O)—T—R^{z}, —NH—T—R^{z}, —O—T—R^{z}, —S—T—R^{z}, —C(O)NH—T—R^{z}, C(O)O—T—R^{z}, C(O)S—T—R^{z}, C(O)NH—T—O—T—R^{z}, —O—T—R^{z}, —T—O—T—R^{z}, —T—S—T—R^{z}, or

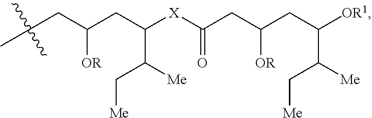

wherein
X is —O—, —NR—, or T—R^{z}1
T is a covalent bond or a bivalent C₁₋₂₆ saturated or unsaturated, straight, or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
two R on the same nitrogen atom are taken with the nitrogen atom to form a 4-7-membered heterocyclic ring having 1-2 heteroatoms independently selected from the group consisting of nitrogen, oxygen, and sulfur.

In one aspect, the present application provides compounds of Formula II:

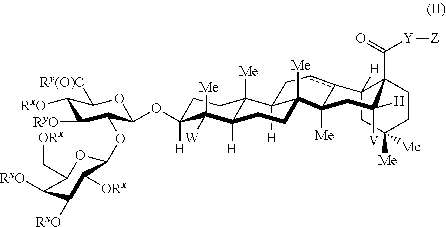

or a pharmaceutically acceptable salt thereof, wherein
[symbol] is a single or double bond;
W is Me, —CHO, or

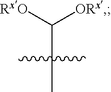

V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

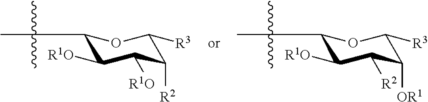

wherein each occurrence of R¹ is R^{x} or a carbohydrate domain having the structure:

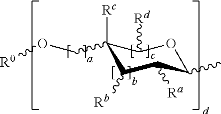

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is —T—R^{z}, —C(O)—T—R^{z}, —NH—T—R^{z}, —O—T—R^{z}, —S—T—R^{z}, —C(O)NH—T—R^{z}, C(O)O—T—R^{z}, C(O)S—T—R^{z}, C(O)NH—T—O—T—R^{z}, —O—T—R^{z}, —T—O—T—R^{z}, —T—S—T—R^{z}, or

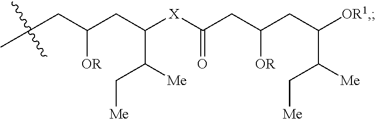

wherein
X is —O—, —NR—, or T—R^{z};
T is a covalent bond or a bivalent C₁₋₂₆ saturated or unsaturated, straight, or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
R^{y} is —OH, —OR, or a carboxyl protecting group selected from the group consisting of ester, amides, and hydrazides; R^{s} is

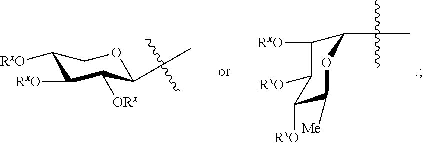

each occurrence of R^{x′} is independently an optionally substituted group selected from 6-10-membered aryl, C₁₋₆ aliphatic, or C₁₋₆ heteroaliphatic having 1-2 heteroatoms independently selected from the group consisting of nitrogen, oxygen, and sulfur; or:
   two R^{x′} are taken together to form a 5-7-membered heterocyclic ring having 1-2 heteroatoms independently selected from the group consisting of nitrogen, oxygen, and sulfur;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur.

It will be appreciated by one of ordinary skill in the art that the compounds of the present application include, but are not necessarily limited to, those compounds encompassed in the genus set forth herein, as well as the compounds described in the detailed description appearing further below. The compounds encompassed by this application include at least all of the compounds disclosed in the entire specification as a whole, including all individual species within each genus.

According to another aspect of the present subject matter, the compounds and formulations disclosed in this application have been shown to be useful as adjuvants. In another aspect, the present application provides a method for preparing compounds according to the embodiments of this application. In another aspect, the present invention provides a method of potentiating an immune response to an antigen, comprising administering to a subject a provided vaccine in an effective amount to potentiate the immune response of said subject to said antigen.

In another aspect, the present invention provides methods of vaccinating a subject, comprising administering a provided vaccine to said subject. In some embodiments, the subject is human. In some embodiments, the vaccine is administered as an injectable.

In another aspect, the invention provides pharmaceutical compositions comprising compounds of the invention and pharmaceutically acceptable excipients. In certain embodiments, the pharmaceutical composition is a vaccine comprising an antigen, adjuvant, and cyclodextrin.

In another aspect, the invention provides kits comprising pharmaceutical compositions of inventive compounds. In some embodiments, the kits comprise prescribing information. In some embodiments, such kits include the combination of an inventive adjuvant compound and another immunotherapeutic agent. The agents may be packaged separately or together. The kit optionally includes instructions for prescribing the medication. In certain embodiments, the kit includes multiple doses of each agent. The kit may include sufficient quantities of each component to treat a subject for a week, two weeks, three weeks, four weeks, or multiple months. In certain embodiments, the kit includes one cycle of immunotherapy. In certain embodiments, the kit includes a sufficient quantity of a pharmaceutical composition to immunize a subject against an antigen long term.

In another aspect, the application provides formulations containing saponin derivates, and salt forms thereof. In certain embodiments, the solvent selected for the formulation may include water, alcohols (including but not limited to methanol, ethanol, butanol, etc.), polyols (including but not limited to glycerol, propylene glycol, polyethylene glycol, etc.), and suitable mixtures thereof, vegetable oils, such as olive oil, and injectable organic esters, such as ethyl oleate.

In certain embodiments, the formulation is a buffered solution having a pH near physiological pH, i.e., between approximately 7.0 and 7.5, between approximately 7.0 and 8.0, between approximately 7.5 and 8.0, or approximately 7.4. In certain embodiments, the buffer is a carbonate-bicarbonate, citrate, acetate, histidine, glycine, phosphate, or tris(hydroxymethyl)aminomethane (Tris or tromethamine) buffer. In certain embodiments, the formulation contains a cyclodextrin. Certain preferred embodiments of the present application include HP-β-CD (2-hydroxypropyl beta-cyclodextrin) or SBECD (sulfobutylether-β-cyclodextrin). In certain embodiments, the excipients prevent precipitation of dissolved saponin derivatives, including TQL-1055, when these solutions have or are brought to physiological pH values. In certain embodiments, certain excipients provide synergistic effects.

In another aspect, the application provides formulations of compositions according to the present application in an adjuvant system. In some embodiments, the adjuvant system utilizes a carrier. In some embodiments, the carrier is a particulate carrier such as metallic salt particles, emulsions, polymers, liposomes, or immune stimulating complexes (ISCOMs). In some embodiments, the adjuvant system includes GLA, MPL, 3D-MPL, LPS, cholesterol, CpG (e.g. CpG 7907 or CpG 1018), PolylC:LC, aluminum hydroxide, aluminum phosphate, tocopherol, acylated monosaccharides, other saponin derivatives (e.g. Quil-A, ISCOM, QS-21, AS02 and ASO1), soluble triterpene glycosides, Toll-like receptor 4 (TLR4) agonists, Toll-like receptor 3 (TLR3) agonists, Toll-like receptor 9 (TLR9) agonists, montanides (ISA51, ISA720), immunostimulatory oligonucleotides, and imidazoquinolines. In some embodiments, the adjuvant system includes known immunostimulants. In some embodiments, the adjuvant system utilizes common adjuvants such as alum, Freund's adjuvant (an oil-in-water emulsion with dead mycobacteria), Freund's adjuvant with MDP (an oil-in-water emulsion with muramyl dipeptide, MDP, a constituent of mycobacteria), alum plus Bordetella pertussis (aluminum hydroxide gel with killed B. pertussis), enterobacteria, FU glycosides, synthetic or derived other membrane vesicles, chitosan microparticles and microcarrier parties, or other known adjuvants.

As used herein, the following definitions shall apply unless otherwise indicated. “Liposomes” as used herein refer to closed bilayer membranes containing an entrapped aqueous volume. Liposomes may also be uni-lamellar vesicles possessing a single membrane bilayer or multi-lamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Liposomes, as they are ordinarily used, consist of smectic mesophases, and can consist of either phospholipid or nonphospholipid smectic mesophases. Smectic mesophase is most accurately described by Small, HANDBOOK OF LIPID RESEARCH, Vol. 4, Plenum, NY, 1986, pp. 49-50. According to Small, “[w]hen a given molecule is heated, instead of melting directly into an isotropic liquid, it may instead pass through intermediate states called mesophases or liquid crystals, characterized by residual order in some directions but by lack of order in others . . . In general, the molecules of liquid crystals are somewhat longer than they are wide and have a polar or aromatic part somewhere along the length of the molecule. The molecular shape and the polar-polar, or aromatic, interaction permit the molecules to align in partially ordered arrays . . . These structures characteristically occur in molecules that possess a polar group at one end. Liquid crystals with long-range order in the direction of the long axis of the molecule are called smectic, layered, or lamellar liquid crystals . . . In the smectic states the molecules may be in single or double layers, normal or tilted to the plane of the layer, and with frozen or melted aliphatic chains.”

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-12 aliphatic carbon atoms. In some embodiments, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR+ (as in N-substituted pyrrolidinyl)).

The term “unsaturated,” as used herein, means that a moiety has one or more units of unsaturation.

As used herein, the term “bivalent C₁₋₁₂ (or C₁₋₂₆, C₁₋₁₆, C₁₋₈) or saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 30, from 1 to 28, from 1 to 26, from 1 to 24, from 1 to 22, from 1 to 20, from 1 to 18, from 1 to 16, from 1 to 14, from 1 to 12, from 1 to 10, from 1 to 8, from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “alkynylene” refers to a bivalent alkynyl group. A substituted alkynylene chain is a polymethylene group containing at least one double bond in which one or more hydrogen atoms are replaced with a substituent. Suitable substituents include those described below for a substituted aliphatic group.

The term “acyl,” used alone or a part of a larger moiety, refers to groups formed by removing a hydroxy group from a carboxylic acid.

The term “halogen” means F, Cl, Br, or I.

The terms “aralkyl” and “arylalkyl” are used interchangeably and refer to alkyl groups in which a hydrogen atom has been replaced with an aryl group. Such groups include, without limitation, benzyl, cinnamyl, and dihyrocinnamyl.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also, included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl.

The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The terms “heteroaralkyl” and “heteroarylalkyl” refer to an alkyl group substituted by a heteroaryl moiety, wherein the alkyl and heteroaryl portions independently are optionally substituted.

The term “heteroaliphatic,” as used herein, means aliphatic groups wherein one or two carbon atoms are independently replaced by one or more of oxygen, sulfur, nitrogen, or phosphorus. Heteroaliphatic groups may be substituted or unsubstituted, branched or unbranched, cyclic or acyclic, and include “heterocycle,” “heterocyclyl,” “heterocycloaliphatic,” or “heterocyclic” groups.

As used herein, the terms “heterocycle,” “heterocyclyl,” “heterocyclic radical,” and “heterocyclic ring” are used interchangeably and refer to a stable 5—to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

In another aspect, the present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration by injection.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio.

Unless otherwise stated, structures depicted herein are also meant to include all isomeric (e.g., enantiomeric, diastereomeric, and geometric (or conformational)) forms of the structure; for example, the R and S configurations for each stereocenter, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Provided compounds may comprise one or more saccharide moieties. Unless otherwise specified, both D- and L-configurations, and mixtures thereof, are within the scope of the invention. Unless otherwise specified, both α- and β-linked embodiments, and mixtures thereof, are contemplated by the present invention.

If, for instance, a particular enantiomer of a compound of the present invention is desired, it may be prepared by asymmetric synthesis, chiral chromatography, or by derivation with a chiral auxiliary, where the resulting diastereomeric mixture is separated and the auxiliary group cleaved to provide the pure desired enantiomers.

Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention.

One of ordinary skill in the art will appreciate that the synthetic methods, as described herein, utilize a variety of protecting groups. By the term “protecting group,” as used herein, it is meant that a particular functional moiety, e.g., O, S, or N, is masked or blocked, permitting, if desired, a reaction to be carried out selectively at another reactive site in a multifunctional compound. In preferred embodiments, a protecting group reacts selectively in good yield to give a protected substrate that is stable to the projected reactions; the protecting group is preferably selectively removable by readily available, preferably non-toxic reagents that do not attack the other functional groups; the protecting group forms a separable derivative (more preferably without the generation of new stereogenic centers); and the protecting group will preferably have a minimum of additional functionality to avoid further sites of reaction. As detailed herein, oxygen, sulfur, nitrogen, and carbon protecting groups may be utilized. By way of non-limiting example, hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl(p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2—or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)-amine, quaternary ammonium salts, N-benzylamine, N-di(4-methoxyphenyl)methylamine, N-5-dibenzosuberylamine, N-triphenylmethylamine 20 (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N′,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), P-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described by Greene and Wuts (supra).

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R⁰; —(CH₂)₀₋₄OR⁰; —O(CH₂)₀₋₄R⁰, —O—(CH₂)₀₋₄C(O)OR⁰; —(CH₂)₀₋₄CH(OR⁰)₂; —(CH₂)₀₋₄SR⁰; —(CH₂)₀₋₄Ph, which may be substituted with R⁰; —(CH₂)₀₋₄O(CH₂)₀₋₁Ph, which may be substituted with R⁰; —CH═CHPh, which may be substituted with R⁰; —(CH₂)₀₋₄ O(CH₂)₀₋₁-pyridyl which may be substituted with R⁰; —NO₂; —CN; —N₃; —(CH₂)₀₋₄N(R⁰)₂; —(CH₂)₀₋₄N(R⁰)C(O)R⁰; —N(R⁰)C(S)R⁰; —(CH₂)₀₋₄N(R⁰)C(O)NR⁰₂; —N(R⁰)C(S)NR⁰₂; —(CH₂)₀₋₄N(R⁰)C(O)OR⁰; —N(R⁰)N(R⁰)C(O)R⁰; —N(R⁰)N(R⁰)C(O)NR⁰₂; —N(R⁰)N(R⁰)C(O)OR⁰; —(CH₂)₀₋₄C(O)R⁰; —C(S)R⁰; —(CH₂)₀₋₄C(O)OR⁰; —(CH₂)₀₋₄C(O)SR⁰; —(CH₂)₀₋₄C(O)OSiR⁰₃; —(CH₂)₀₋₄OC(O)R⁰; —OC(O)(CH₂)₀₋₄SR, —SC(S)SR⁰; —(CH₂)₀₋₄SC(O)R⁰; —(CH₂)₀₋₄C(O)NR⁰₂; —C(S)NR⁰₂; —C(S)SR⁰; —SC(S)SR⁰, —(CH₂)₀₋₄OC(O)NR⁰₂; —C(O)N(OR⁰)R⁰; —C(O)C(O)R⁰; —C(O)CH₂C(O)R⁰; —C(NOR⁰)R⁰; —(CH₂)₀₋₄SSR⁰; —(CH₂)₀₋₄S(O)₂R⁰; —(CH₂)₀₋₄S(O)₂OR⁰; —(CH₂)₀₋₄O S(O)₂R⁰; —S(O)₂NR⁰₂; —(CH₂)₀₋₄S(O)R⁰; —N(R⁰)S(O)₂NR⁰₂; —N(R⁰)S(O)₂R⁰; —N(OR⁰)R⁰; —C(NH)NR⁰₂; —P(O)₂R⁰; —P(O)R⁰₂; —OP(O)R⁰₂; —OP(O)(OR⁰)₂; SiR⁰₃; —(C₁₋₄ straight or branched)alkylene)O—N(R⁰)₂; or —(C₁₋₄ straight or branched)alkylene)C(O)O—N(R⁰)₂, wherein each R⁰ may be substituted as defined below and is independently hydrogen, C₁₋₆ aliphatic, —CH₂Ph, —O(CH₂)₀₋₁Ph, —CH₂—(5-6-membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R⁰, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R⁰ (or the ring formed by taking two independent occurrences of R⁰ together with their intervening atoms), are independently halogen, —(CH₂)₀₋₂R^{Δ}, -(haloR^{Δ}), —(CH₂)₀₋₂OH, —(CH₂)₀₋₂OR^{Δ}, —(CH₂)₀₋₂CH(OR^{A})₂; —O(haloR^{Δ}), —CN, —N₃, —(CH₂)₀₋₂C(O)R^{Δ}, —(CH₂)₀₋₂C(O)OH, —(CH₂)₀₋₂C(O)OR^{Δ}, —(CH₂)₀₋₂SR^{Δ}, —(CH₂)₀₋₂SH, —(CH₂)₀₋₂NH₂, —(CH₂)₀₋₂NHR^{Δ}, —(CH₂)₀₋₂NR^{Δ}, —NO₂, —SiR^{Δ}₃, —OSiR^{Δ}₃, —C(O)SR^{Δ}, —(C₁₋₄ straight or branched alkylene)C(O)OR^{Δ}, or —SSR. wherein each R^{Δ} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R⁰ include ═0 and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, -R^{Δ}, —(haloR^{Δ}), —OH, —OR^{Δ}, —O(haloR^{Δ}), —CN, —C(O)OH, —C(O)OR^{Δ}, —NH₂, —NHR^{Δ}, —NR⁰₂, or —NO₂, wherein each R^{Δ} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur. Suitable substituents on the aliphatic group of R^{†} are independently halogen, —R^{Δ}, —(haloR^{Δ}), —OH, —OR^{Δ}, —O(haloR^{Δ}), —CN, —C(O)OH, —C(O)OR^{Δ}, —NH₂, —NHR^{Δ}, —NR⁰₂, or —NO₂, wherein each R^{Δ} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug, or other material other than directly into the central nervous system, such that it enters the patient's system and, thus, is subject to metabolism and other like processes, for example, subcutaneous administration.

The term “enriched” as used herein refers to a mixture having an increased proportion of one or more species. In some embodiments, the mixture is “enriched” following a process that increases the proportion of one or more desired species in the mixture. In some embodiments, the desired species comprise(s) greater than 10% of the mixture. In some embodiments, the desired species comprise(s) greater than 25% of the mixture. In some embodiments, the desired species comprise(s) greater than 40% of the mixture. In some embodiments, the desired species comprise(s) greater than 60% of the mixture. In some embodiments, the desired species comprise(s) greater than 75% of the mixture. In some embodiments, the desired species comprise(s) greater than 85% of the mixture. In some embodiments, the desired species comprise(s) greater than 90% of the mixture. In some embodiments, the desired species comprise(s) greater than 95% of the mixture. Such proportions can be measured any number of ways, for example, as a molar ratio, volume to volume, or weight to weight.

The term “pure” refers to compounds that are substantially free of compounds of related non-target structure or chemical precursors (when chemically synthesized). This quality may be measured or expressed as “purity.” In some embodiments, a target compound has less than about 30%, 20%, 10%, 5%, 2%, 1%, 0.5%, and 0.1% of non-target structures or chemical precursors. In certain embodiments, a pure compound of present invention is only one prosapogenin compound (i.e., separation of target prosapogenin from other prosapogenins).

The term “carbohydrate” refers to a sugar or polymer of sugars. The terms “saccharide”, “polysaccharide”, “carbohydrate”, and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

Further objects, features, and advantages of the present application will become apparent form the detailed which is set forth below when considered together with the figures of drawing.

### BRIEF DESCRIPTION OF THE DRAWINGS

FIGS. 1 to 3 depict anti-gE total IgG endpoint titers for embodiments discussed in Example 4.

FIGS. 4 to 6 depict the survival rates for each formulation for each mouse strain as discussed in Example 5.

FIGS. 7 to 9 depict weight loss data observed for each mouse strai as discussed in Example 5.

FIGS. 10 to 12 depict eight loss and survival data post dose 1 as discussed in Example 6.

FIGS. 13 to 15 depict eight loss and survival data post dose 2 as discussed in Example 6.

FIG. 16 depicts anti-HA total IgG endpoint titers post dose 2 for each group as discussed in Example 6.

FIG. 17 depicts animal weight data after dose 1 as discussed in Example 7.

FIG. 18 depicts animal weight data after dose 2 as discussed in Example 7.

FIGS. 19 to 25 depict anti-gE total IgG endpoint titers post dose 2 as discussed in Example 7.

### DETAILED DESCRIPTION OF CERTAIN EMBODIMENTS

### Compounds

Compounds of this invention include those described generally above, and are further illustrated by the classes, subclasses, and species disclosed herein. In some embodiments, provided compounds are analogs of naturally occurring triterpene glycoside saponins and intermediates thereto. For purposes of this invention, the chemical elements are identified in accordance with the Periodic Table of the Elements, CAS version, Handbook of Chemistry and Physics, 75th Ed. Additionally, general principles of organic chemistry are described in Organic Chemistry, Thomas Sorrell, University Science Books, Sausalito: 1999, and March's Advanced Organic Chemistry, 5th Ed., Ed.: Smith, M. B. and March, J., John Wiley & Sons, New York: 2001, the entire contents of which are hereby incorporated by reference.

In some embodiments, provided compounds are analogs of Quillaja saponins. In some embodiments, provided compounds are prosapogenins. In certain embodiments, provided compounds are analogs of QS-7 and QS-21 and possess potent adjuvant activity.

In one aspect, the present application provides compounds of Formula I:

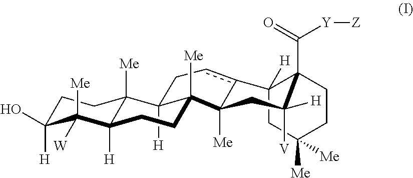

or a pharmaceutically acceptable salt thereof, wherein
[symbol] is a single or double bond;
W is —CHO;
V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

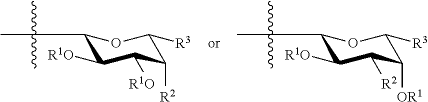

wherein each occurrence of R¹ is R^{x} or a carbohydrate domain having the structure:

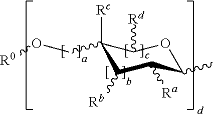

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is —T—R^{z}, —C(O)—T—R^{z}, —NH—T—R^{z}, —O—T—R^{z}, —S—T—R^{z}, —C(O)NH—T—R^{z}, C(O)O—T—R^{z}, C(O)S—T—R^{z}, C(O)NH—T—O—T—R^{z}, —O—T—R^{z}, —T—O—T—R^{z}, —T—S—T—R^{z}, or

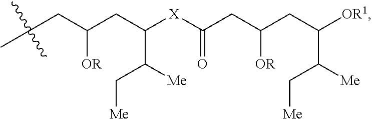

wherein
X is —O—, —NR—, or T—R^{z}.
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
two R on the same nitrogen atom are taken with the nitrogen atom to form a 4-7-membered heterocyclic ring having 1-2 heteroatoms independently selected from the group consisting of nitrogen, oxygen, and sulfur.

In one aspect, the present application provides compounds of Formula II:

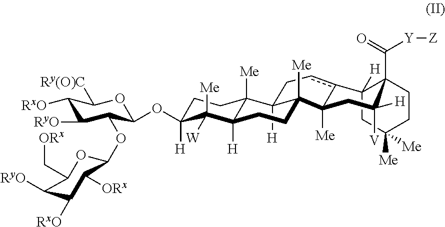

or a pharmaceutically acceptable salt thereof, wherein
[symbol] is a single or double bond;
W is Me, —CHO, or

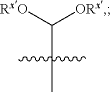

V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

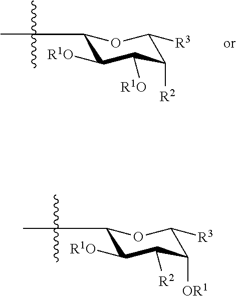

wherein each occurrence of R¹ is R^{x} or a carbohydrate domain having the structure:

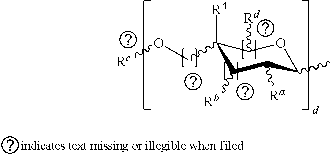

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is —T—R^{z}, —C(O)—T—R^{z}, —NH—T—R^{z}, —O—T—R^{z}, —S—T—R^{z}, —C(O)NH—T—R^{z}, C(O)O—T—R^{z}, C(O)S—T—R^{z}, C(O)NH—T—O—T—R^{z}, —O—T—R^{z}, —T—O—T—R^{z}, —T—S—T—R^{z}, or

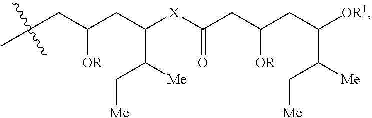

wherein
X is —O—, —NR—, or T—R^{z}.
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
R^{y} is —OH, —OR, or a carboxyl protecting group selected from the group consisting of ester, amides, and hydrazides;
R^{s} is

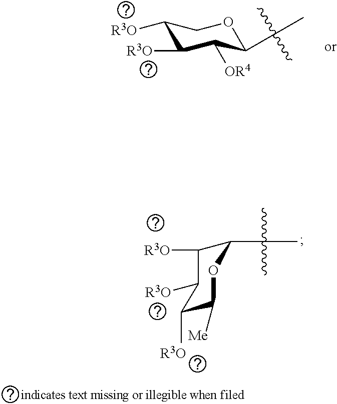

each occurrence of R^{x} is independently an optionally substituted group selected from 6-10-membered aryl, C₁₋₆ aliphatic, or C₁₋₆ heteroaliphatic having 1-2 heteroatoms independently selected from the group consisting of nitrogen, oxygen, and sulfur; or:
   two R^{x′} are taken together to form a 5-7-membered heterocyclic ring having 1-2 heteroatoms independently selected from the group consisting of nitrogen, oxygen, and sulfur;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur.

In one aspect, the present application provides compounds of Formula I:

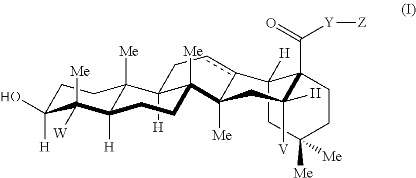

or a pharmaceutically acceptable salt thereof, wherein
[symbol] is a single or double bond;
W is —CHO;
V is —OH;
Y is —O—;
wherein Z is a carbohydrate domain having the structure:

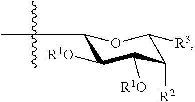

wherein:
R¹ is independently H or

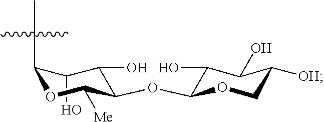

R² is NHR⁴;
R³ is CH₂OH; and
R⁴ is —T—R^{z}, —C(O)—T—R^{z}, —NH—T—R^{z}, —O—T—R^{z}, —S—T—R^{z}, —C(O)NH—T—R^{z}, C(O)O—T—R^{z}, C(O)S—T—R^{z}, C(O)NH—T—O—T—R^{z}, —O—T—R^{z}, —T—O—T—R^{z}, —T—S—T—R^{z}, or

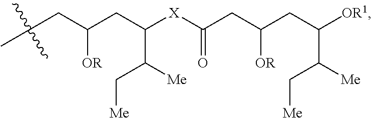

wherein:
X is —O—, —NR—, or T—R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur.

It will be appreciated by one of ordinary skill in the art that the compounds of the present application include but are not necessarily limited to those compounds encompassed in the genus definitions set forth as part of the present section. The compounds encompassed by this application include at least all of the compounds disclosed in the entire specification as a whole, including all individual species within each genus.

In certain embodiments, V is OR^{x}. In certain embodiments V is OH. In certain embodiments, V is H.

In certain embodiments, Y is —O—. In certain embodiments, Y is —NH—. In certain embodiments, Y is —NR—. In certain embodiments, Y is CH₂.

In certain embodiments, Z is hydrogen. In certain embodiments, Z is a cyclic or acyclic, optionally substituted moiety. In certain embodiments, Z is an acyl. In certain embodiments, Z is an aliphatic. In certain embodiments, Z is a heteroaliphatic. In certain embodiments, Z is aryl. In certain embodiments Z is arylalkyl. In certain embodiments, Z is heteroacyl. In certain embodiments, Z is heteroaryl. In certain embodiments, Z is a carbohydrate domain having the structure:

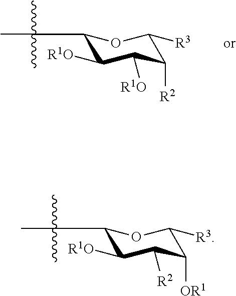

In some embodiments Z is a carbohydrate domain having the structure:

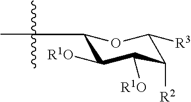

wherein:
R¹ is independently H or

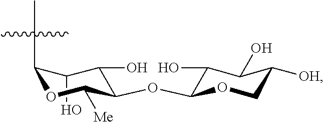

R² is NHR⁴,
R³ is CH₂OH, and
R⁴ is selected from:

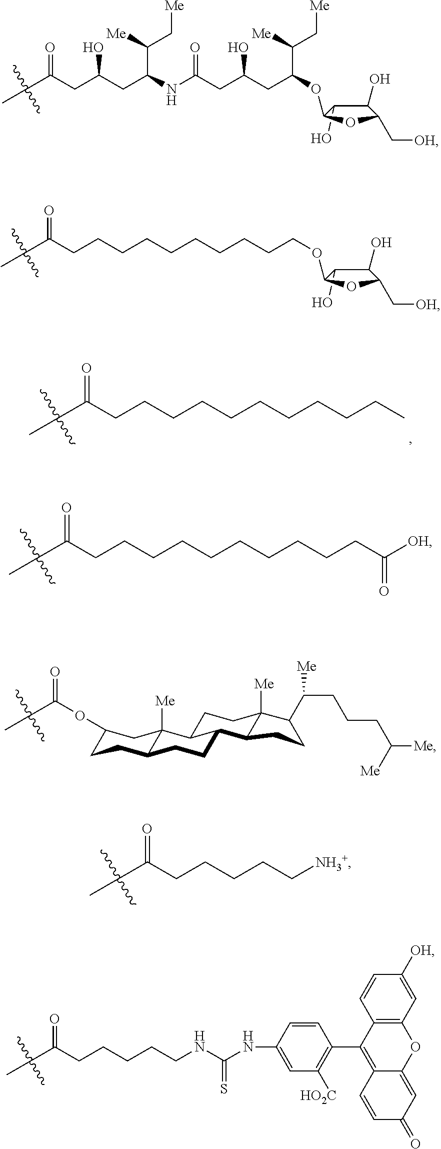

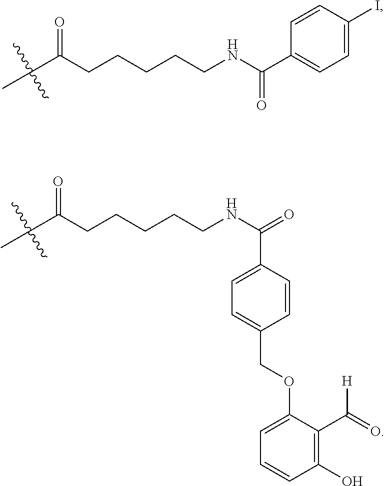 [SMILES: *C(=O)CCCCCNC(=O)c1ccc(I)cc1 ; *C(=O)CCCCCNC(=O)c1ccc(COc2cccc(O)c2C=O)cc1]

In some embodiments, R¹ is R^{x}. In other embodiments, R¹ a carbohydrate domain having the structure:

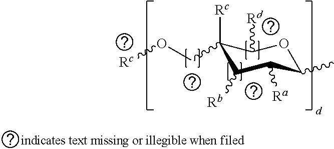

In some aspects, each occurrence of a, b, and c is independently 0, 1, or 2. In some embodiments, d is an integer from 1-5. In some embodiments, each d bracketed structure may be the same. In some embodiments, each d bracketed structure may be different. In some embodiments, the d bracketed structure represents a furanose or a pyranose moiety. In some embodiments, and the sum of b and c is 1 or 2.

In some embodiments, R⁰ is hydrogen. In some embodiments, R⁰ is an oxygen protecting group selected from the group. In some embodiments, R⁰ is an alkyl ether. In some embodiments, R⁰ is a benzyl ether. In some embodiments, R⁰ is a silyl ether. In some embodiments, R⁰ is an acetal. In some embodiments, R⁰ is ketal. In some embodiments, R⁰ is an ester. In some embodiments, R⁰ is a carbamate. In some embodiments, R⁰ is a carbonate. In some embodiments, R⁰ is an optionally substituted moiety. In some embodiments, R⁰ is an acyl. In some embodiments, R⁰ is a C₁₋₁₀ aliphatic. In some embodiments, R⁰ is a C₁₋₆ heteroaliphatic. In some embodiments, R⁰ is a 6-10-membered aryl. In some embodiments, R⁰ is an arylalkyl. In some embodiments, R⁰ is a 5-10 membered heteroaryl having 1-4 heteroatoms independently selected from nitrogen, oxygen, or sulfur. In some embodiments, R⁰ is a 4-7 membered heterocyclyl having 1-2 heteroatoms independently selected from the group consisting of nitrogen, oxygen, and sulfur.

In some embodiments, R^{a} is hydrogen. In some embodiments, R^{a} is a halogen. In some embodiments, R^{a} is OH. In some embodiments, R^{a} is OR. In some embodiments, R^{a} is OR^{x}. In some embodiments, R^{a} is NR₂. In some embodiments, R^{a} is NHCOR. In some embodiments, R^{a} an acyl. In some embodiments, R^{a} is C₁₋₁₀ aliphatic. In some embodiments, R^{a} is C₁₋₆ heteroaliphatic. In some embodiments, R^{a} is 6-10-membered aryl. In some embodiments, R^{a} is arylalkyl. In some embodiments, R^{a} is 5-10-membered heteroaryl having 1-4 heteroatoms independently selected from nitrogen, oxygen, sulfur. In some embodiments, R^{a} is 4-7-membered heterocyclyl having 1-2 heteroatoms independently selected from the group consisting of nitrogen, oxygen, and sulfur.

In some embodiments, R^{b} is hydrogen. In some embodiments, R^{b} is a halogen. In some embodiments, R^{b} is OH. In some embodiments, R^{b} is OR. In some embodiments, R^{b} is OR^{x}. In some embodiments, R^{b} is NR₂. In some embodiments, R^{b} is NHCOR. In some embodiments, R^{b} an acyl. In some embodiments, R^{b} is C₁₋₁₀ aliphatic. In some embodiments, R^{b} is C₁₋₆ heteroaliphatic. In some embodiments, R^{b} is 6-10-membered aryl. In some embodiments, R^{b} is arylalkyl. In some embodiments, R^{b} is 5-10-membered heteroaryl having 1-4 heteroatoms independently selected from nitrogen, oxygen, sulfur. In some embodiments, R^{b} is 4-7-membered heterocyclyl having 1-2 heteroatoms independently selected from the group consisting of nitrogen, oxygen, and sulfur.

In some embodiments, R^{b} is hydrogen. In some embodiments, R^{b} is a halogen. In some embodiments, R^{b} is OH. In some embodiments, R^{b} is OR. In some embodiments, R^{b} is OR^{x}. In some embodiments, R^{b} is NR₂. In some embodiments, R^{b} is NHCOR. In some embodiments, R^{b} an acyl. In some embodiments, R^{b} is C₁₋₁₀ aliphatic. In some embodiments, R^{b} is C₁₋₆ heteroaliphatic. In some embodiments, R^{b} is 6-10-membered aryl. In some embodiments, R^{b} is arylalkyl. In some embodiments, Rb is 5-10-membered heteroaryl having 1-4 heteroatoms independently selected from nitrogen, oxygen, sulfur. In some embodiments, R^{b} is 4-7-membered heterocyclyl having 1-2 heteroatoms independently selected from the group consisting of nitrogen, oxygen, and sulfur.

In some embodiments, R^{c} is hydrogen. In some embodiments, R^{c} is a halogen. In some embodiments, R^{c} is OH. In some embodiments, R^{c} is OR. In some embodiments, R^{c} is OR^{x}. In some embodiments, R^{c} is NR₂. In some embodiments, R^{c} is NHCOR. In some embodiments, R^{c} an acyl. In some embodiments, R^{c} is C₁₋₁₀ aliphatic. In some embodiments, R^{c} is C₁₋₆ heteroaliphatic. In some embodiments, R^{c} is 6-10-membered aryl. In some embodiments, R^{c} is arylalkyl. In some embodiments, R¹ is 5-10-membered heteroaryl having 1-4 heteroatoms independently selected from nitrogen, oxygen, sulfur. In some embodiments, R^{c} is 4-7-membered heterocyclyl having 1-2 heteroatoms independently selected from the group consisting of nitrogen, oxygen, and sulfur.

In some embodiments, R^{d} is hydrogen. In some embodiments, R^{d} is a halogen. In some embodiments, R^{d} is OH. In some embodiments, R^{d} is OR. In some embodiments, R^{d} is OR^{x}. In some embodiments, R^{d} is NR₂. In some embodiments, R^{d} is NHCOR. In some embodiments, R^{d} an acyl. In some embodiments, R^{d} is C₁₋₁₀ aliphatic. In some embodiments, R^{d} is C₁₋₆ heteroaliphatic. In some embodiments, R^{d} is 6-10-membered aryl. In some embodiments, R^{d} is arylalkyl. In some embodiments, R^{d} is 5-10-membered heteroaryl having 1-4 heteroatoms independently selected from nitrogen, oxygen, sulfur. In some embodiments, R^{d} is 4-7-membered heterocyclyl having 1-2 heteroatoms independently selected from the group consisting of nitrogen, oxygen, and sulfur.

In some embodiments, R² is hydrogen. In some embodiments, R² is a halogen. In some embodiments, R² is OH. In some embodiments, R² is OR. In some embodiments, R² is OC(O)R⁴. In some embodiments, R² is OC(O)OR⁴. In some embodiments, R² is OC(O)NHR⁴. In some embodiments, R² is OC(O)NRR⁴. In some embodiments, R² is OC(O)SR⁴. In some embodiments, R² is NHC(O)R⁴. In some embodiments, R² is NRC(O)R⁴. In some embodiments, R² is NHC(O)OR⁴. In some embodiments, R² is NHC(O)NHR⁴. In some embodiments, R² is NHC(O)NRR⁴. In some embodiments, R² is NHR⁴. In some embodiments, R² is N(R⁴)₂. In some embodiments, R² is NHR⁴ In some embodiments, R² is NRR⁴. In some embodiments, R² is N₃. In some embodiments, R² is C₁₋₁₀ aliphatic. In some embodiments, R² is C₁₋₆ heteroaliphatic. In some embodiments, R² is 6-10-membered aryl. In some embodiments, R² is arylalkyl. In some embodiments, R² is 5-10 membered heteroaryl having 1-4 heteroatoms independently selected from the group consisting of nitrogen, oxygen, and sulfur. In some embodiments, R² is 4-7-membered heterocyclyl having 1-2 heteroatoms independently selected from the group consisting of nitrogen, oxygen, and sulfur.

In some embodiments, R³ is hydrogen. In some embodiments, R³ is a halogen. In some embodiments, R³ is CH₂OR¹. In some embodiments, R³ is an acyl. In some embodiments, R³ is C₁₋₁₀ aliphatic. In some embodiments, R³ is C₁₋₆ heteroaliphatic. In some embodiments, R³ is 6-10-membered aryl. In some embodiments, R³ is arylalkyl. In some embodiments, R³ is 5-10-membered heteroaryl having 1-4 heteroatoms independently selected from the group consisting of nitrogen, oxygen, and sulfur. In some embodiments, R³ is 4-7-membered heterocyclyl having 1-2 heteroatoms independently selected from the group consisting of nitrogen, oxygen, and sulfur.

In some embodiments, R⁴ is —T—R^{z}. In some embodiments, R⁴ is —C(O)—T—R^{z}. In some embodiments, R⁴ is —NH—T—R^{z}. In some embodiments, R⁴ is —O—T—R^{z}. In some embodiments, R⁴ is —S—T—R^{z}. In some embodiments, R⁴ is —C(O)NH—T—R^{z}. In some embodiments, R⁴ is C(O)O—T—R^{z}. In some embodiments, R⁴ is C(O)S—T—R^{z}. In some embodiments, R⁴ is C(O)NH—T—O—T—R^{z}. In some embodiments, R⁴ is —O—T—R^{z}. In some embodiments, R⁴ is —T—O—T—R^{z}. In some embodiments, R⁴ is —T—S—T—R^{z}. In some embodiments, R⁴ is

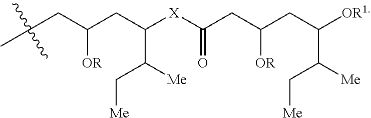

In some embodiments, X is —O—. In some embodiments, X is —NR—. In some embodiments, X is T—R^{z}.

In some embodiments, T is a covalent bond or a bivalent C₁₋₂₆ saturated or unsaturated, straight or branched, aliphatic or heteroaliphatic chain.

In some embodiments, R^{z} is hydrogen. In some embodiments, R^{z} is a halogen. In some embodiments, R^{z} is —OR. In some embodiments, R^{z} is —OR^{x}. In some embodiments, R^{z} is —OR¹. In some embodiments, R^{z} is —OR″. In some embodiments, R^{z} is —SR. In some embodiments, R^{z} is NR₂. In some embodiments, R^{z} is —C(O)OR. In some embodiments, R^{z} is —C(O)R. In some embodiments, R^{z} is —NHC(O)R. In some embodiments, R^{z} is —NHC(O)OR. In some embodiments, R^{z} is NC(O)OR. In some embodiments, R^{z} is an acyl. In some embodiments, R^{z} is arylalkyl. In some embodiments, R^{z} is heteroarylalkyl. In some embodiments, R^{z} is C₁₋₆ aliphatic. In some embodiments, R^{z} is 6-10-membered aryl. In some embodiments, R^{z} is 5-10-membered heteroaryl having 1-4 heteroatoms independently selected from nitrogen, oxygen, or sulfur. In some embodiments, R^{z} is 4-7-membered heterocyclyl having 1-2 heteroatoms independently selected from the group consisting of nitrogen, oxygen, and sulfur.

In some embodiments, R^{x} is hydrogen. In some embodiments, R^{x} is an oxygen protecting group. In some embodiments, R^{x} is an alkyl ether. In some embodiments, R^{x} is a benzyl ether. In some embodiments, R^{x} is silyl ether. In some embodiments, R^{x} is an acetal. In some embodiments, R^{x} is ketal. In some embodiments, R^{x} is ester. In some embodiments, R^{x} is carbamate. In some embodiments, R^{x} is carbonate.

In some embodiments, R^{y} is —OH. In some embodiments, R^{y} is —OR. In some embodiments, R^{y} is a carboxyl protecting group. In some embodiments, R^{y} is an ester. In some embodiments, R^{y} is an amide. In some embodiments, R^{y} is a hydrazide.

In some embodiments, R^{S} is

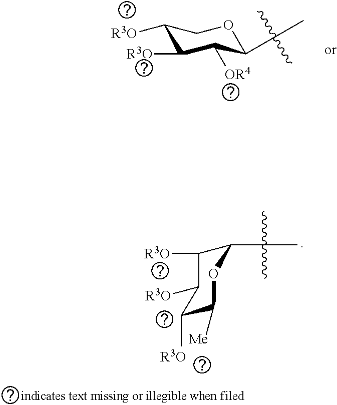

In some embodiments, R^{x′} is optionally substituted 6-10-membered aryl. In some embodiments, R^{x′} is optionally substituted C₁₋₆ aliphatic. In some embodiments, R^{x′} is optionally substituted or C₁₋₆ heteroaliphatic having 1-2 heteroatoms independently selected from the group consisting of nitrogen, oxygen, and sulfur. In some embodiments, two R^{x′} are taken together to form a 5-7-membered heterocyclic ring having 1-2 heteroatoms independently selected from the group consisting of nitrogen, oxygen, and sulfur.

In some embodiments, R is hydrogen. In some embodiments, R is an acyl. In some embodiments, R is arylalkyl. In some embodiments, R is 6-10-membered aryl. In some embodiments, R is C₁₋₆ aliphatic. In some embodiments, R is C₁₋₆ heteroaliphatic having 1-2 heteroatoms independently selected from the group consisting of nitrogen, oxygen, and sulfur. In some embodiments, two R on the same nitrogen atom are taken with the nitrogen atom to form a 4-7-membered heterocyclic ring having 1-2 heteroatoms independently selected from the group consisting of nitrogen, oxygen, and sulfur.

In some embodiments, R¹ has the same embodiments as R¹.

Exemplary compounds of Formula I are set forth in Table 1 below:

TABLE 1
EXEMPLARY COMPOUNDS OF FORMULA I

| | |
|---|---|
| 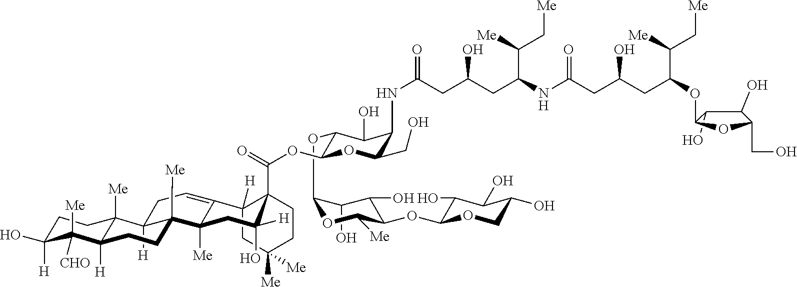 | I-1 |
| 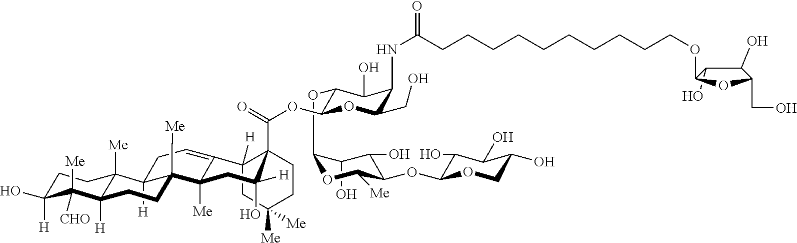 | I-2 |
| 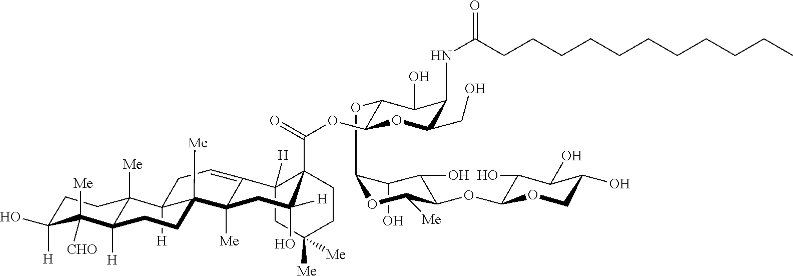 | I-3 |
| 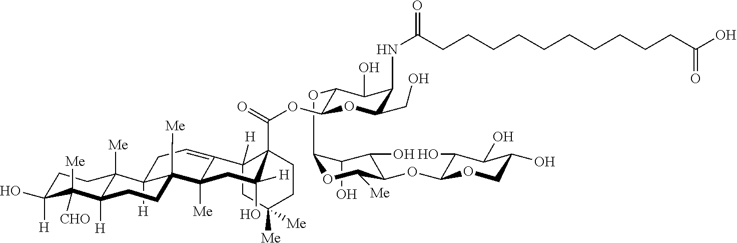 | I-4 |
| 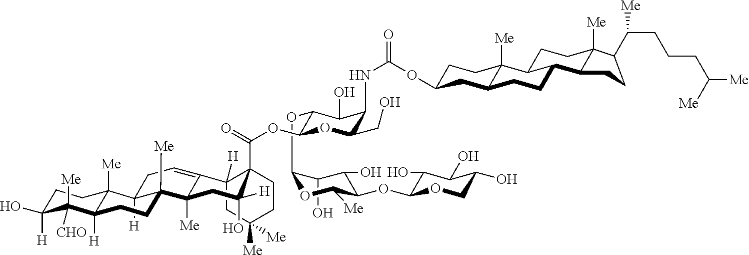 | I-5 |
| 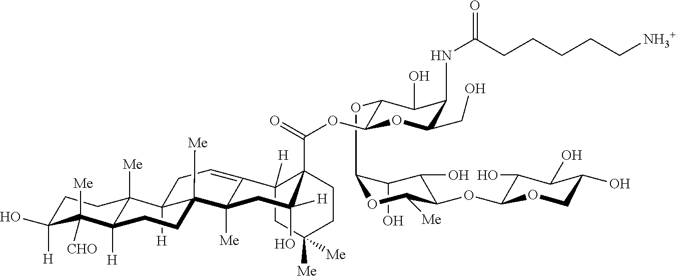 | I-6 |
| 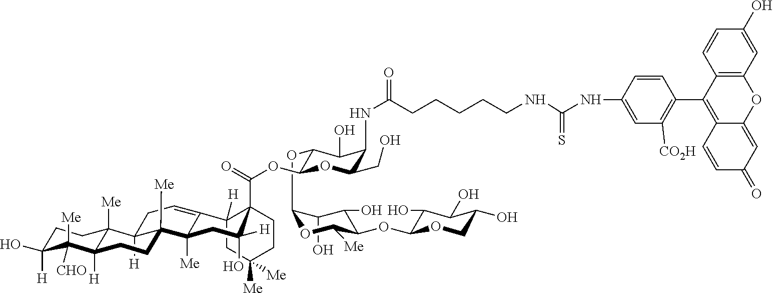 | I-7 |
| 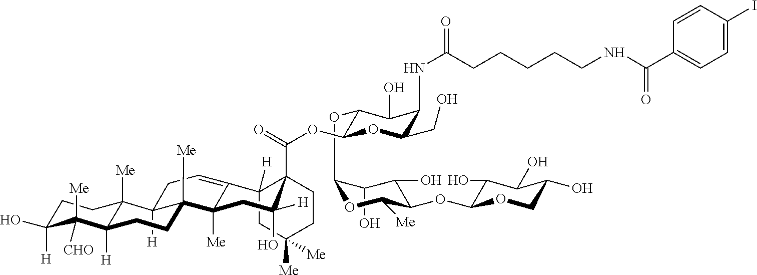 | I-8 |
| 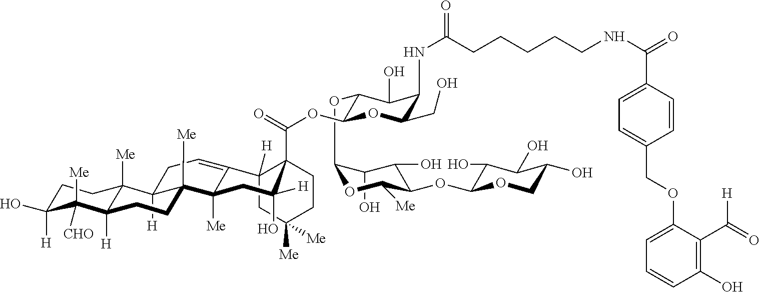 | I-9 |
| 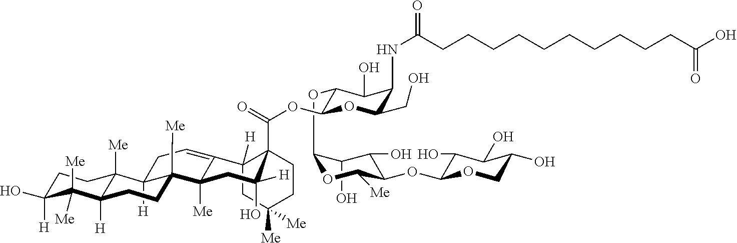 | I-10 |

It will be appreciated that it is not an object of the present subject matter to claim compounds disclosed in the prior art that are the result of isolation or degradation studies on naturally occurring prosapogenins or saponins.

### Synthesis

The compounds of the present application may be synthesized as provided in PCT/US2009/039954, PCT/US2015/33567, PCT/US2016/67530, PCT/US2016/60564, and/or PCT/US2018/027462.

### Adjuvants

The present application encompasses the recognition that synthetic access to and structural modification of QS-21 and related Quillaja saponins may afford compounds with high adjuvant potency and low toxicity, as well as having more stability and being more cost effective. Accordingly, compounds of the present application, including TQL-1055, have industrial applicability and are useful as adjuvants, in free form acid or base form or in pharmaceutically acceptable salt form.

### Salt Forms

Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, β-toluenesulfonate, undecanoate, valerate salts, and the like. In some embodiments, the counterion is selected from chloride, sulfate, bromide, mesylate, maleate, citrate, nitrate, tosylate, tartrate, phosphate, acetate, camsylate, formate, fumarate, oxalate, thiocyanate, adipate, caprate, caproate, caprylate, dodecylsulfate, glutarate, laurate, oleate, palmitate, sebacate, stearate, undecylenate, iodide, choline (e.g. choline hydroxide), L-lysine, sodium (e.g. sodium carbonate or sodium hydroxide), calcium (e.g. calcium hydroxide), potassium (e.g. potassium carbonate), magnesium (e.g. magnesium hydroxide), meglumine, ammonium, trimethylamine, triethylamine, ethanolamine, diethanolamine, triethanolamine, 1-Pyrrolidineethanol, trimethyltetradecylammonium (e.g. trimethyltetradecylammonium-hydroxide), tetraethanol-ammonium (e.g. tetraethanol-ammonium hydroxide), procaine, benzathine, aluminum, zinc, piperazine, tromethamine, diethylamine, ethylenediamine, arginine, histidine, glycine, lithium tetrakis(pentafluorophenyl)borate, tetraphenylboranuide, hexafluorophosphate, tetrafluoroborate, bis(triphenylphosphine)iminium chloride, tetraphenylphosphonium chloride, tetra-n-butylammonium bromide (TBAB), alkali metals bound by crown ethers, and mixtures thereof. In some embodiments, the counterion is a quaternary ammonium salt.

In other cases, the compounds of the present invention may contain one or more acidic functional groups and, thus, are capable of forming pharmaceutically-acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, tertiary, or quaternary amine. Salts derived from appropriate bases include alkali metal, alkaline earth metal, ammonium, and N+(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Representative organic amines useful for the formation of base addition salts include ethylamine, diethylamine, ethylenediamine, ethanolamine, diethanolamine, piperazine and the like.

Further pharmaceutically acceptable salts include, when appropriate, choline, L-lysine, magnesium, meglumine, potassium, sodium, arginine, histidine, and TEA.

Certain embodiments of the present application include salt forms of synthetic saponin-derived adjuvants. In some embodiments, the adjuvants are compounds of Formula I as described herein. In some embodiments, the adjuvant is the compound TQL-1055.

In another preferred embodiment of the present application, a choline salt form of TQL-1055 is provided as Form A, as described in PCT/US2021/023591, incorporated herein by reference.

### Vaccines

Compositions in this application and their pharmaceutically acceptable salts are useful as vaccines to induce active immunity towards antigens in subjects. Any animal that may experience the beneficial effects of the compositions of the present application is within the scope of subjects that may be treated. In some embodiments, the subjects are mammals. In some embodiments, the subjects are humans.

The vaccines of the present application may be used to confer resistance to infection by either passive or active immunization. When the vaccines of the present application are used to confer resistance through active immunization, a vaccine of the present application is administered to an animal to elicit a protective immune response which either prevents or attenuates a proliferative or infectious disease.

When the vaccines of the present application are used to confer resistance to infection through passive immunization, the vaccine is provided to a host animal (e.g., human, dog, or mouse), and the antisera elicited by this vaccine is recovered and directly provided to a recipient suspected of having an infection or disease or exposed to a causative organism.

The present application thus concerns and provides a means for preventing or attenuating a proliferative disease resulting from organisms which have antigens that are recognized and bound by antisera produced in response to the immunogenic antigens included in vaccines of the present application. As used herein, a vaccine is said to prevent or attenuate a disease if its administration to an animal results either in the total or partial attenuation (i.e., suppression) of a symptom or condition of the disease, or in the total or partial immunity of the animal to the disease.

The administration of the vaccine (or the antisera which it elicits) may be for either a “prophylactic” or “therapeutic” purpose. When provided prophylactically, the vaccine(s) are provided in advance of any symptoms of proliferative disease. The prophylactic administration of the vaccine(s) serves to prevent or attenuate any subsequent presentation of the disease. When provided therapeutically, the vaccine(s) is provided upon or after the detection of symptoms which indicate that an animal may be infected with a pathogen. The therapeutic administration of the vaccine(s) serves to attenuate any actual disease presentation. Thus, the vaccines may be provided either prior to the onset of disease proliferation (so as to prevent or attenuate an anticipated infection) or after the initiation of an actual proliferation.

One of ordinary skill in the art will appreciate that vaccines may optionally include a pharmaceutically acceptable excipient or carrier. Thus, according to another aspect, provided vaccines may comprise one or more antigens that are optionally conjugated to a pharmaceutically acceptable excipient or carrier. In some embodiments, said one or more antigens are conjugated covalently to a pharmaceutically acceptable excipient. In other embodiments, said one or more antigens are non-covalently associated with a pharmaceutically acceptable excipient.

As described above, adjuvants may be used to increase the immune response to an antigen. According to the present application, provided vaccines may be used to invoke an immune response when administered to a subject. In certain embodiments, an immune response to an antigen may be potentiated by administering to a subject a provided vaccine in an effective amount to potentiate the immune response of said subject to said antigen.

### Formulations

The compounds of the present application and/or their salts may be combined with one or a mixture of pharmaceutically acceptable excipients and hydrophilic or lipophilic solvents to form a pharmaceutical composition. In certain embodiments, formulations of the present application include injectable formulations. In certain embodiments, the pharmaceutical composition includes a pharmaceutically acceptable amount of a compound of the present application. In certain embodiments, the compounds of the application and an antigen form an active ingredient. The amount of active ingredient(s) which can be combined with a carrier material to produce a single dosage form will vary depending upon the host being treated, and the particular mode of administration. The amount of active ingredient(s) that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%, or from about 1% to 99%, preferably from 10% to 90%, 20% to 80%, 30% to 70%, 40% to 60%, 45% to 55%, or about 50%.

Thus, one aspect of the present application is certain formulations containing salt forms or free forms of saponin derivatives, including free acid and salt forms of TQL-1055. Particularly preferred embodiments of such solutions or suspensions remain relatively stable in liquid form even near physiological pH values without precipitating and without denaturing an antigen present in solution.

At the outset, it is important to note the formulation approaches discussed herein are not necessarily mutually exclusive. Rather, the formulations of the present application may include combinations of different approaches discussed below, e.g., a formulation including liposomes and an emulsion. Furthermore, it should be appreciated that certain portions of the formulation may include some ingredients, whereas others contain other ingredients. For example, a formulation may include liposomes having a toll-like receptor agonist, as well as an emulsion containing a compound of Formula I in the dispersed phase. In other words, the compound of Formula I and the other ingredients need not be present in every part of the formulation. Rather, individual ingredients may be included in individual parts to maximize formulation properties, including efficacy, stability, and pH.

In certain embodiments, the solvent selected for the formulation may include water, alcohols (including but not limited to methanol, ethanol, butanol, etc.), polyols (including but not limited to glycerol, propylene glycol, polyethylene glycol, etc.), and suitable mixtures thereof, vegetable oils, such as olive oil, and injectable organic esters, such as ethyl oleate.

In certain embodiments, the formulation includes a buffered solution having a pH near physiological pH, i.e., between approximately 7.0 and 7.5, between approximately 7.0 and 8.0, between approximately 7.5 and 8.0, or approximately 7.4. In certain embodiments, the buffer is a carbonate-bicarbonate, citrate, acetate, histidine, glycine, phosphate, or tris(hydroxymethyl)aminomethane (Tris or tromethamine) buffer. In certain embodiments, the formulation contains cyclodextrin. In some embodiments, the cyclodextrin is an α-cyclodextrin (αCD), β-cyclodextrin (βCD), or γ-cyclodextrin (γCD). Certain preferred embodiments of the present application include β-cyclodextrins (βCD). Certain particularly preferred embodiments include HP-β-CD (2-hydroxypropyl beta-cyclodextrin) or SBECD (sulfobutylether-β-cyclodextrin).

In certain embodiments, the cyclodextrin has the following chemical formula:

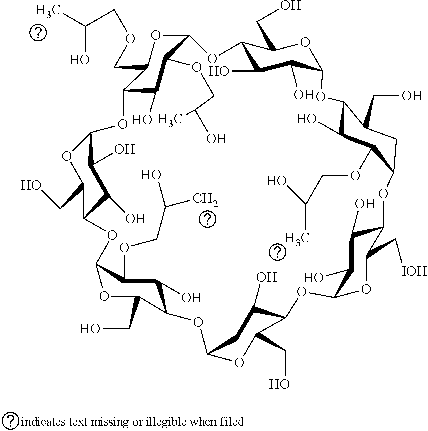

In certain embodiments, the cyclodextrin has the following chemical formula:

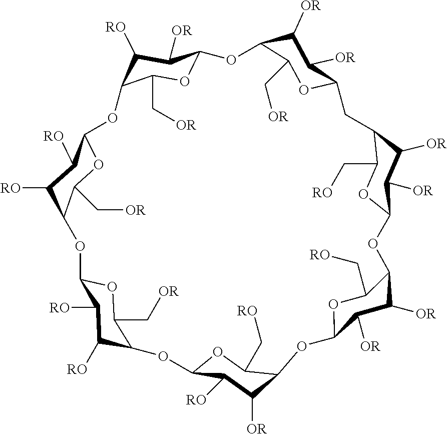

wherein each instance of R is independently H, —(CH₂)ₙ—CH₃ with n from 0 to 6, —(CH₂)ₙ—OH with n from 1 to 6, —(CH₂)ₙ—CHOH—CH₃ with n from 1 to 6, —(CH₂)ₙSO₃—Na⁺ with n from 1 to 6, glycosyl, or maltosyl.

In certain embodiments, the cyclodextrin concentration in solution is approximately 1%, 5%, 10%, 15%, 20%, 25%, or 30%. In certain embodiments, the cyclodextrin concentration is between 5% and 10%. In certain embodiments, the cyclodextrin concentration is between 10% and 15%. In certain embodiments, the cyclodextrin concentration is between 15% and 20%. In certain embodiments, the cyclodextrin concentration is between 20% and 25%. In certain embodiments, the cyclodextrin concentration is between 25% and 30%.

In certain embodiments, the cyclodextrin excipients prevent precipitation of dissolved saponin derivatives, including TQL-1055, when these solutions have or are brought to physiological pH values. In certain embodiments, certain excipients provide synergistic effects. To explore the dose-response of the foregoing stable cyclodextrin formulations, as discussed above and in the examples below, experiments were conducted using a formulation containing an antigen and TQL-1055 FA (free acid) HP-β-CD; however, higher doses of this formulation unexpectedly resulted in substantial mortality in mice. Further follow-on studies demonstrated (1) TQL-1055 administered without HP-β-CD did not result in mortality at any dose tested, and (2) HP-β-CD administered without TQL-1055 did not result in mortality at any dose tested; and (3) certain dose levels of the combination of TQL-1055 and HP-β-CD unexpectedly resulted in murine mortality.

The inventors of the present application have invented certain stable formulations of synthetic saponin molecules, including TQL-1055, which contain cyclodextrins but surprisingly are tolerable in mammals and substantially improve physiochemical and/or biopharmaceutical properties, for example poor solubility in aqueous-based solutions, such that these formulations are useful for widespread (e.g., commercial) pharmaceutical use.

Thus, in certain embodiments, the present application provides stable formulations containing TQL-1055 salt and a cyclodextrin. In a preferred embodiment, the stable formulation of the present application contains TQL-1055 potassium salt and HP-R-CD. In some embodiments, the TQL-1055 potassium salt is an in situ salt.

In some embodiments, the TQL-1055 counterion is choline. In some embodiments, the counterion is a counterion listed below.

In certain embodiments, the formulation includes a compound of Formula I and a counterion. In a preferred embodiment, the compound of formula I is Compound 1-4 and the counterion is choline. Acceptable counterions maintain electric neutrality and potentially increase solubility of the compound of Formula I in solution. In a preferred embodiment, the counterion is a cation. In some embodiments, the counterion is an anion. In some embodiments, the counterion is lipophilic. In some embodiments, the counterion is lipophobic. In some embodiments, the counterion is hydrophilic. In some embodiments, the counterion is hydrophobic. In some embodiments, the counterion is selected from the group consisting of chloride, sulfate, bromide, mesylate, maleate, citrate, nitrate, tosylate, tartrate, phosphate, acetate, camsylate, formate, fumarate, oxalate, thiocyanate, adipate, caprate, caproate, caprylate, dodecylsulfate, glutarate, laurate, oleate, palmitate, sebacate, stearate, undecylenate, iodide, choline (e.g. choline hydroxide), L-lysine, sodium (e.g. sodium carbonate or sodium hydroxide), calcium (e.g. calcium hydroxide), potassium (e.g. potassium carbonate or potassium hydroxide), magnesium (e.g. magnesium hydroxide), meglumine, ammonium, trimethylamine, triethylamine, ethanolamine, diethanolamine, triethanolamine, 1-Pyrrolidineethanol, trimethyltetradecylammonium (e.g. trimethyltetradecylammonium-hydroxide), tetraethanol-ammonium (e.g. tetraethanol-ammonium hydroxide), procaine, benzathine, aluminum, zinc, piperazine, tromethamine, diethylamine, ethylenediamine, arginine, histidine, glycine, lithium tetrakis(pentafluorophenyl)borate, tetraphenylboranuide, hexafluorophosphate, tetrafluoroborate, bis(triphenylphosphine)iminium chloride, tetraphenylphosphonium chloride, tetra-n-butylammonium bromide (TBAB), alkali metals bound by crown ethers, and mixtures thereof. In some embodiments, the counterion is a quaternary ammonium salt. Other pharmaceutically acceptable counterions adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

In certain embodiments, the formulation includes an emulsion. An emulsion is generally a thermodynamically unstable multi-phase system containing immiscible materials, the first of which is relatively uniformly dispersed as globules throughout a second, continuous phase. A third emulsifying agent is added to stabilize the system and prevent the dispersed phase from coalescing and/or precipitating. In some embodiments, the emulsifying agent reduces the interfacial tension between the dispersed phase and the continuous phase. In some embodiments, the emulsifying agent provides a barrier between the dispersed phase and the continuous phase. In some embodiments, the emulsifying agent is a surfactant. Suitable surfactants according to embodiments of the present application are discussed herein. In some embodiments, the emulsifying agent is selected from gum acacia, tween, span, veegum, tragacanth, methylcellulose, saponins, and soaps formed from monovalent bases like Na+, K+, and NH₄+. In some embodiments, the emulsifying agent is wool fat, resins, beeswax, and soaps from divalent bases like Ca+, Mg²⁺, and Zn²⁺. In some embodiments, the emulsifying agent is selected from agar, albumin, alginates, casein, ceatyl alcohol, cholic acid, desoxycholic acid, diacetyl tartaric acid esters, egg yolk, glycerol, triglycerides, gums, irish moss (carrageenan), lecithin, mono- and diglycerides, monosodium phosphate, monostearate, ox bile extract, propylene glycol, soaps, taurocholic acid, and sodium oleate (or its sodium salt).

In some embodiments, the emulsion is an oil-in-water emulsion. In some embodiments, the emulsion is a water-in-oil emulsion. In some embodiments, the emulsion is a multiple emulsion, such as a water-in-oil-in-water emulsion, or an oil-in-water-in-oil emulsion.

In some embodiments, the continuous phase is selected from water, alcohols (including but not limited to methanol, ethanol, butanol, etc.), polyols (including but not limited to glycerol, propylene glycol, polyethylene glycol, etc.), oils, vegetable oils, such as olive oil, injectable organic esters, such as ethyl oleate, and suitable mixtures thereof.

The dispersed phase is generally immiscible with the continuous phase. In some embodiments, the dispersed phase is selected from water, alcohols (including but not limited to methanol, ethanol, butanol, etc.), polyols (including but not limited to glycerol, propylene glycol, polyethylene glycol, etc.), oils, vegetable oils, such as olive oil, injectable organic esters, such as ethyl oleate, and suitable mixtures thereof. In some embodiments, the dispersed phase contains a compound of Formula I or Formula II, preferably Compound I-4. In some embodiments, the dispersed phase is a free form of a compound of Formula I or Formula II. In some embodiments, the dispersed phase is a salt form of a compound of Formula I or Formula II, as discussed herein.

In certain embodiments, the formulation includes a surfactant. In some embodiments, the surfactant is an emulsifying agent, as described above. In some embodiments, the surfactant forms micelles. The structure of a micelle is such that hydrophobic (non-polar) tails of the surfactant are oriented toward the center of the micelle while the hydrophilic (polar) heads orient towards the aqueous phase, which the micelles are in an aqueous solution. In some embodiments, the micelles contain a compound of Formula I, preferably Compound 1-4. In some embodiments, the micelles contain a toll-like receptor agonist, such as a TLR4 agonist. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose, hydroxyethylcellulose, polyethyleneglycol, polyaspartamide and hydrophilic peptide sequences as described in U.S. Pat. Nos. 6,316,024; 6, 126,966; 6,056,973; and 6,043,094. Other acceptable micelle forming compounds include as well PEGylated lipids such as 1,2-distearoyl-sn-glycero-3-phosphoethanolamine-N-[amino(polyethylene glycol)-2000]. Liposomes can be made without hydrophilic polymers. Therefore, liposome formulations may or may not contain hydrophilic polymers.

Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatide acid, phosphatidylserine, phosphatidylglycerol, phosphatidylinositol, and sphingomyelin as disclosed in U.S. Pat. Nos. 6,056,973 and 5,874,104.

The vesicle-forming lipids may also be glycolipids, cerebrosides, or cationic lipids, such as 1,2-dioleoyl-sn-glycero-3-phosphocholine (DOPC), 1,2-dioleoyl-sn-glycero-3-phospho-(1′-rac-glycerol) (DOPG), 1,2-dipalmitoyl-sn-glycero-3-phospho-(1′-rac-glycerol) (DPPG), 1,2-dioleoyl-sn-glycero-3-phospho-L-serine (DOPS), L-α-phosphatidylserine (Brain, Porcine) (Brain PS), 1,2-dimyristoyl-sn-glyero-3-phospho-(1′rac-glycerol) (DMPG), 1,2-dioleoyl-sn-glycero-3-phosphoethanol (Phosphatidylethanol), L-α-phosphatidic acid (Egg, Chicken) (Egg PA), DDA (dimethyldioctadecylammonium), DC-cholesterol (3B-[N-(N′,N′-dimethylaminoethane)-carbamoyl]cholesterol), PS (phosphatidylserine), PA (1,2-dioleoyl-sn-glycero-3-phosphate), POPG (1-palmitoyl-2-oleoyl-sn-glycero-3-phospho-(1′-rac-glycerol)), PC (phosphatidylcholine), DSPC (1,2-distearoyl-sn-glycero-3-phosphocholine), DLPC (1,2-dilauroyl-sn-glycero-3-phosphocholine), DPPC (1,2-dipalmitoyl-sn-glycero-3-phosphocholine), dimyristoyl phosphatidylcholine (DMPC), 1,2-dioleyloxy-3-(trimethylamino)propane (DOTAP); N-[1-(2,3,-ditetradecyloxy)propyl]-N,N-dimethyl-N-hydroxyethylammonium bromide (DMRIE); N-[I [(2,3,-dioleyloxy)propyl]-N,N-dimethyl-N-hydroxy ethylammonium bromide (DORIE); N-[I-(2,3-dioleyloxy)propyl]-N,N,N-trimethylammonium chloride (DOTMA); 3 [N-(N′,N′-dimethylaminoethane) carbamoly] cholesterol (DCChol); or dimethyldioctadecylammonium (DDAB) also as disclosed in U.S. Pat. No. 6,056,973. Cholesterol may also be present in the proper range to impart stability to the liposome vesicle, as disclosed in U.S. Pat. Nos. 5,916,588 and 5,874,104.

Additional liposomal technologies are described in U.S. Pat. Nos. 6,759,057; 6,406,713; 6,352,716; 6,316,024; 6,294,191; 6,126,966; 6,056,973; 6,043,094; 5,965,156; 5,916,588; 5,874,104; 5,215,680; and 4,684,479. These described liposomes and lipid-coated microbubbles, and methods for their manufacture. Thus, one skilled in the art, considering both the present disclosure and the disclosures of these other patents could produce a liposome for the purposes of the present embodiments. Liposomes may comprise phospholipid or nonphospholipid bilayers.

Phospholipid bilayers may comprise hydrocarbon chains, optionally having a melting temperature in water of at least 23° C. Such phospholipids may comprise, for example, dimyristoyl phosphatidylcholine (DMPC), dimyristoyl phosphatidylglycerol (DMPG), cholesterol (Chol), or similar molecules, and mixtures thereof, The liposome may optionally comprise a neutral lipid that is non-crystalline at room temperature, such as dioleoyl phosphatidylcholine or similar compounds. See U.S. Published Patent Application No. 2011/0206758.

During manufacture of liposomal formulations containing, for example, QS-21, small unicellular liposomal vesicles (SUV) are first created. The SUV is then added to an aqueous environment having QS-21 or another saponin and the SUV takes up QS-21 or the saponin from the aqueous environment. The liposomal composition also may have certain optional ingredients, such as for example MPL, synthetic MPL such as MPLA, CpG 7909 or CpO 1018, or similar substances.

In some embodiments, the liposomes are formed using the thin-film hydration method. Such technique involves creating a thin film by removing an organic solvent containing lipids/cholesterol, and, upon adding and agitating a dispersion medium, heterogeneous liposomes are formed. The heterogeneous mixture may be extruded through a membrane to obtain homogeneous small liposomes. Thus, in some embodiments, a compound of Formula I, preferably Compound 1-4, or a salt thereof, and lipids/cholesterol are dissolved in methanol:chloroform solvent, dried, and then hydrated in a buffer (e.g., PBS) to form liposomes containing the compound of Formula 1. In some embodiments, the liposomes are formed by combining a lipid such as a cholesterol and methanol in the presence of a compound of Formula I, preferably Compound 1-4, or a salt thereof. In such an embodiment, the liposomes are then added to an aqueous environment having, for example, MPL or other compositions as set forth above.

Thus, in one aspect the present application provides formulations comprising a liposome formulation of MPL and Compound 1-4, In another aspect the present application provides formulations comprising MPL, Compound 1-4, and a squalene emulsion. In another aspect the present application provides formulations comprising MPL, Compound 1-4, and CpG 7909 or CpG 1018. MPL is a heterogeneous mixture of molecules from a biological source including both agonists and antagonists for TLR4. CpG 7909 is an immunomodulating synthetic oligonucleotide designed to specifically agonise the Toll-like receptor 9 (TLR9).

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Non-limiting examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Non-limiting examples of suitable aqueous and nonaqueous carriers, which may be employed in the pharmaceutical compositions of the present application include water, alcohols (including but not limited to methanol, ethanol, butanol, etc.), polyols (including but not limited to glycerol, propylene glycol, polyethylene glycol, etc.), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain additives such as preservatives, Netting agents, emulsifying agents, and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a formulation, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form.

Regardless of the route of administration selected, the compounds of the present application, which may be used in a suitable hydrated form, and/or the pharmaceutical compositions of the present application, are formulated into pharmaceutically-acceptable dosage forms by conventional methods known to those of skill in the art.

Actual dosage levels of the active ingredients in the pharmaceutical compositions of the present application may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present application employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the present application employed in the pharmaceutical composition at levels lower than that required to achieve the desired therapeutic effect and then gradually increasing the dosage until the desired effect is achieved.

In some embodiments, a compound or pharmaceutical composition of the present application is provided to a subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition of the present application repeatedly over the life of the subject. Preferred chronic treatments involve regular administrations, for example one or more times a day, one or more times a week, or one or more times a month. In general, a suitable dose, such as a daily dose of a compound of the present application, will be that amount of the compound that is the lowest dose effective to produce a therapeutic effect. Such an effective dose will generally depend upon the factors described above.

Generally, doses of the compounds of the present application for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

In some embodiments, provided adjuvant compounds of the present application are administered as pharmaceutical compositions or vaccines, In certain embodiments, it is contemplated that the amount of adjuvant compound administered will be 1-2000 μg. In certain embodiments, it is contemplated that the amount of adjuvant compound administered will be 1-1000 μg. In certain embodiments, it is contemplated that the amount of adjuvant compound administered will be 1-500 μg. In certain embodiments, it is contemplated that the amount of adjuvant compound administered will be 1-250 μg. In certain embodiments, it is contemplated that the amount of adjuvant compound administered will be 100-1000 μg. In certain embodiments, it is contemplated that the amount of adjuvant compound administered will be 100-500 μg. In certain embodiments, it is contemplated that the amount of adjuvant compound administered will be 100-200 μg. In certain embodiments, it is contemplated that the amount of adjuvant compound administered will be 250-500 μg.

In certain embodiments, it is contemplated that the amount of adjuvant compound administered will be 10-1000 μg. In certain embodiments, it is contemplated that the amount of adjuvant compound administered will be 500-1000 μg. In certain embodiments, it is contemplated that the amount of adjuvant compound administered will be 50-250 μg. In certain embodiments, it is contemplated that the amount of adjuvant compound administered will be 50-500 μg.

In some embodiments, provided adjuvant compounds of the present application are administered as pharmaceutical compositions or vaccines. In certain embodiments, it is contemplated that the amount of adjuvant compound administered will be 1-2000 mg. In certain embodiments, it is contemplated that the amount of adjuvant compound administered will be 1-1000 mg. In certain embodiments, it is contemplated that the amount of adjuvant compound administered will be 1-500 mg.

In certain embodiments, it is contemplated that the amount of adjuvant compound administered will be 1-250 mg. In certain embodiments, it is contemplated that the amount of adjuvant compound administered will be 100-1000 mg. In certain embodiments, it is contemplated that the amount of adjuvant compound administered will be 100-500 mg. In certain embodiments, it is contemplated that the amount of adjuvant compound administered will be 100-200 mg. In certain embodiments, it is contemplated that the amount of adjuvant compound administered will be 250-500 mg.

In certain embodiments, it is contemplated that the amount of adjuvant compound administered will be 10-1000 mg. In certain embodiments, it is contemplated that the amount of adjuvant compound administered will be 500-1000 mg. In certain embodiments, it is contemplated that the amount of adjuvant compound administered will be 50-250 mg. In certain embodiments, it is contemplated that the amount of adjuvant compound administered will be 50-500 mg. In certain embodiments, it is contemplated that the amount of adjuvant compound administered will be 0.01-215.4 mg.

In certain embodiments, it is contemplated that the amount of adjuvant administered will be 1000-5000 μg/kg. In certain embodiments, it is contemplated that the amount of adjuvant administered will be 1000-4000 μg/kg. In certain embodiments, it is contemplated that the amount of adjuvant administered will be 1000-3000 μg/kg.

In certain embodiments, it is contemplated that the amount of adjuvant administered will be 1000-2000 μg/kg. In certain embodiments, it is contemplated that the amount of adjuvant administered will be 2000-5000 μg/kg. In certain embodiments, it is contemplated that the amount of adjuvant administered will be 2000-4000 μg/kg. In certain embodiments, it is contemplated that the amount of adjuvant administered will be 2000-3000 μg/kg. In certain embodiments, it is contemplated that the amount of adjuvant administered will be 3000-5000 μg/kg. In certain embodiments, it is contemplated that the amount of adjuvant administered will be 3000-4000 μg/kg. In certain embodiments, it is contemplated that the amount of adjuvant administered will be 4000-5000 μg/kg. In certain embodiments, it is contemplated that the amount of adjuvant administered will be 1-500 μg/kg. In certain embodiments, it is contemplated that the amount of adjuvant administered will be 500-1000 μg/kg. In certain embodiments, it is contemplated that the amount of adjuvant administered will be 1000-1500 μg/kg. In certain embodiments, it is contemplated that the amount of adjuvant administered will be 1 mg/kg. In certain embodiments, it is contemplated that the amount of adjuvant administered will be 2 mg/kg. In certain embodiments, it is contemplated that the amount of adjuvant administered will be 3 mg/kg. In certain embodiments, it is contemplated that the amount of adjuvant administered will be 4 mg/kg. In certain embodiments, it is contemplated that the amount of adjuvant administered will be 5 mg/kg. In certain embodiments, it is contemplated that the amount of adjuvant administered will be 0.0029-5 mg/kg. In certain embodiments, the amount of adjuvant administered in females is less than the amount of adjuvant administered in males. In certain embodiments, the amount of adjuvant administered to infants is less than the amount of adjuvant administered to adults. In certain embodiments, the amount of adjuvant administered to pediatric recipients is less than the amount of adjuvant administered to adults. In certain embodiments, the amount of adjuvant administered to immunocompromised recipients is more than the amount of adjuvant administered to healthy recipients. In certain embodiments, the amount of adjuvant administered to elderly recipients is more than the amount of adjuvant administered to non-elderly recipients.

If desired, the effective dose of the active compound may be administered as two, three, four, five, six or more sub-doses administered separately at appropriate intervals throughout the day, optionally, in unit dosage forms.

While it is possible for a compound of the present application to be administered alone, in certain embodiments the compound is administered as a pharmaceutical formulation or composition as described above.

The compounds according to the present application may be formulated for administration in any convenient way for use in human or veterinary medicine, by analogy with other pharmaceuticals.

### Kits

The present application provides kits comprising pharmaceutical formulations or compositions of a compound of the present application. In some embodiments, the kits include two or more vials (or other pharmaceutically acceptable vessels) that must be combined prior to administration. In some embodiments, a first vial contains an antigen component, which may be in solution (in a formulation) or may be lyophilized, e.g., a powder. In some embodiments, a second vial contains an adjuvant formulation, which may be in an aqueous or lyophilized form, for example a formulation according to the present application. Such adjuvant formulations include compounds of Formula I, preferably Compound 1-4, or a salt thereof. Such adjuvant formulations may include multiple formulation approaches, as discussed above. For example, the second vial may include an adjuvant formulation (e.g., Compound 1-4) and a surfactant.

In some embodiments, the first vial contains a formulation including a compound of Formula I, preferably Compound 1-4, or a salt thereof, and the second vial contains a surfactant, or a salt thereof. The first or second vial (or a third vial) may also contain an antigen and other excipients.

Some embodiments include a third vial having a TLR agonist, e.g., a TLR4 agonist formulation.

The vials may be packaged together or separately, In some embodiments, the kit includes instructions for combining the vials and administering the combined formulation to a patient in need thereof.

The kit optionally includes instructions for prescribing the medication. In certain embodiments, the kit includes multiple doses of each agent. The kit may include sufficient quantities of each component to treat one or more subject for a week, two weeks, three weeks, four weeks, or multiple months. The kit may include a full cycle of immunotherapy. In some embodiments, the kit includes a vaccine comprising one or more bacterial or viral-associated antigens, and one or more provided compounds.

### Methods

The present application also encompasses methods of conferring immune resistance to an individual. Such methods include administering to an individual a vaccine comprising a therapeutically effective amount of a compound of Formula I or Formula II, in free form or in pharmaceutically acceptable salt form, together with an antigen. In particular, the compound of Formula I or Formula II may be TQL-1055, preferably in formulation with cyclodextrin, for example HP-β-CD.

The present application also encompasses methods of formulation compounds of Formula I, or salts thereof, according to the present application. A person of ordinary skill in the art would understand how to prepare the formulations discussed herein based on the above disclosure of the present application.

### Further Embodiments

1.1. A pharmaceutical formulation comprising
a cyclodextrin; and
a pharmaceutically acceptable salt of a compound of Formula I

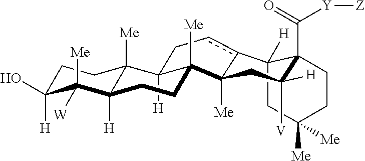

wherein
[symbol] is a single or double bond;
W is —CHO;
V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

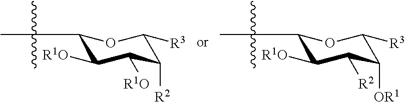

wherein each occurrence of R¹ is R^{x} or a carbohydrate domain having the structure:

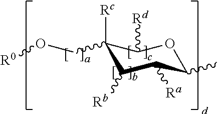

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is —T—R^{z}, —C(O)—T—R^{z}, —NH—T—R^{z}, —O—T—R^{z}, —S—T—R^{z}, —C(O)NH—T—R^{z}, C(O)O—T—R^{z}, C(O)S—T—R^{z}, C(O)NH—T—O—T—R^{z}, —O—T—R^{z}, —T—O—T—R^{z}, —T—S—T—R^{z}, or

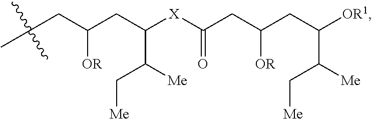

wherein
X is —O—, —NR—, or T—R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
two R on the same nitrogen atom are taken with the nitrogen atom to form a 4-7-membered heterocyclic ring having 1-2 heteroatoms independently selected from the group consisting of nitrogen, oxygen, and sulfur.
1.2. The pharmaceutical formulation of 1.1, wherein the compound of Formula I is Compound 1-4:

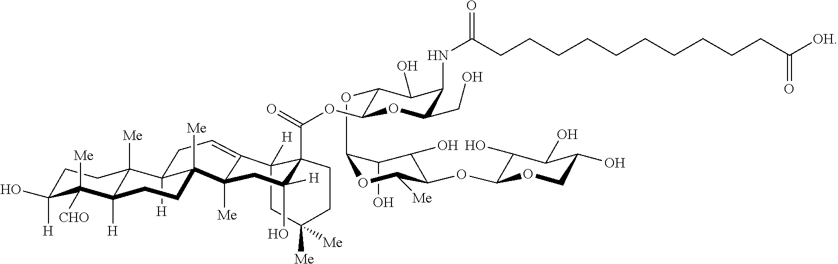

1.3. The pharmaceutical formulation of 1.1 to 1.2,
wherein the cyclodextrin is selected from the group consisting of α-cyclodextrins (αCD), β-cyclodextrins (βCD), and γ-cyclodextrin (γCD).
1.4. The pharmaceutical formulation of 1.1 to 1.3, wherein the cyclodextrin is a β-cyclodextrin (βCD).
1.5 The pharmaceutical formulation of 1.1 to 1.4,
wherein the cyclodextrin is selected from the group consisting of HP-β-CD (2-hydroxypropyl beta-cyclodextrin) and SBECD (sulfobutylether-β-cyclodextrin).
1.6 The pharmaceutical formulation of 1.1 to 1.5, wherein the cyclodextrin is HP-β-CD (2-hydroxypropyl beta-cyclodextrin).
1.7 The pharmaceutical formulation of 1.1 to 1.2, wherein the cyclodextrin is of formula

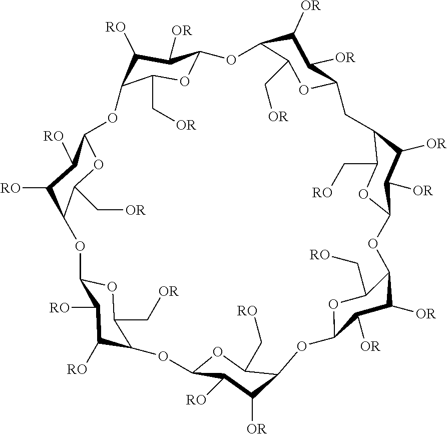

wherein each instance of R is independently H, —(CH₂)ₙ—CH₃ with n from 0 to 6, —(CH₂)ₙ—OH with n from 1 to 6, —(CH₂)ₙ—CHOH—CH₃ with n from 1 to 6, —(CH₂)ₙSO₃—Na⁺ with n from 1 to 6, glycosyl, or maltosyl.
1.8. The pharmaceutical formulation of 1.1 to 1.7, further comprising an antigen.
1.9. The pharmaceutical formulation of 1.8, wherein the antigen is associated with a bacteria or virus.
1.10. The pharmaceutical formulation of 1.9, wherein the antigen is associated with SARS-CoV-2 virus.
1.11. The pharmaceutical formulation of 1.9, wherein the antigen is associated with Varicella Zoster.
1.12 The pharmaceutical formulation of 1.1 to 1.11, wherein the pharmaceutical formulation is stable at physiological pH for at least 12 months.
1.13 The pharmaceutical formulation of 1.1 to 1.11, wherein the pharmaceutical formulation is stable at physiological pH for at least 24 months.
1.14 The pharmaceutical formulation of 1.1 to 1.11, wherein the pharmaceutical formulation is stable at physiological pH for at least 36 months.
1.15 The pharmaceutical formulation of 1.12 to 1.14, wherein physiological pH is between approximately 7.0 and 7.5.
1.16 The pharmaceutical formulation of 1.12 to 1.14, wherein physiological pH is between approximately 7.0 and 8.0.
1.17 The pharmaceutical formulation of 1.12 to 1.14, wherein physiological pH between approximately 7.5 and 8.0.
1.18 The pharmaceutical formulation of 1.12 to 1.14, wherein physiological pH is approximately 7.4.
1.19 The pharmaceutical formulation of 1.1 to 1.18, wherein the pharmaceutically acceptable salt is a potassium salt.
1.20 A method of conferring resistance to an infection, the method comprising administering an antigen in combination with a pharmaceutical formulation of 1.1 to 1.19.
2.1. A liquid formulation comprising a pharmaceutically acceptable salt of Compound 1-4:

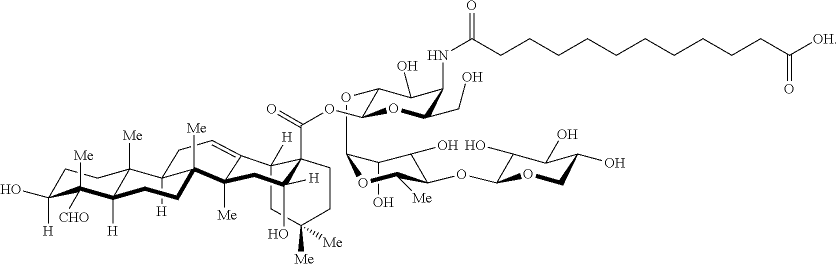

2.2. The liquid formulation of 2.1, wherein the liquid formulation comprises a solvent selected from the group consisting of water, methanol, and ethanol.
2.3. The liquid formulation of 2.1, further comprising a buffer selected from the group consisting of carbonate-bicarbonate, citrate, acetate, histidine, glycine, phosphate, or tris(hydroxymethyl)aminomethane (Tris or tromethamine) buffer.
2.4. The liquid formulation of 2.1, further comprising a cyclodextrin.
2.5. The liquid formulation of 2.4, wherein the cyclodextrin is selected from the group consisting of α-cyclodextrins (αCD), β-cyclodextrins (βCD), and γ-cyclodextrin (γCD).
2.6. The liquid formulation of 2.4, wherein the cyclodextrin is a β-cyclodextrin (βCD).
2.7 The liquid formulation of 2.4, wherein the cyclodextrin is selected from the group consisting of HP-β-CD (2-hydroxypropyl beta-cyclodextrin) and SBECD (sulfobutylether-β-cyclodextrin).
2.8 The liquid formulation of 2.4, wherein the cyclodextrin is HP-β-CD (2-hydroxypropyl beta-cyclodextrin).
2.9 The liquid formulation ion of 1.1 to 1.2, wherein the cyclodextrin is of formula

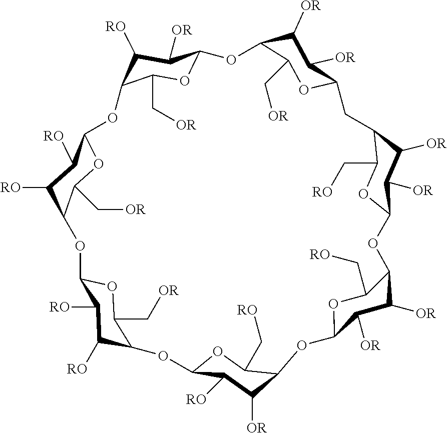

wherein each instance of R is independently H, —(CH₂)ₙ—CH₃ with n from 0 to 6, —(CH₂)ₙ—OH with n from 1 to 6, —(CH₂)ₙ—CHOH—CH₃ with n from 1 to 6, —(CH₂)ₙSO₃—Na⁺ with n from 1 to 6, glycosyl, or maltosyl.
2.10. The liquid formulation of 2.1 to 2.9, further comprising an antigen.
2.11. The liquid formulation of 2.10, wherein the antigen is associated with a bacteria, virus, protozoa, or fungus.
2.12. The liquid formulation of 2.10, wherein the antigen is associated with SARS-CoV-2 virus.
2.13. The liquid formulation of 2.10, wherein the antigen is associated with Varicella Zoster.
2.14 The liquid formulation of 2.1 to 2.13, wherein the pharmaceutically acceptable salt is a potassium salt.
2.14. A method of conferring resistance to an infection, the method comprising administering the liquid formulation of 2.1 to 2.14.

### EXAMPLES

### Example 1—Formulation of TQL-1055 in HP-β-CD solution

Two solutions were prepared: (1) 15% w/v HP-β-CD in 1 ml of PBS, add TQL-1055 (crushed) to achieve 1 mg/ml; and (2) 20% w/v HP-β-CD in 1 ml of PBS; add TQL-1055 (crushed) to achieve 1 mg/ml. Both solutions were solubilized using heat and sonication for one hour at 60° C. TQL-1055 dissolved completely in both solutions.

Optical density shown below. Clarity is defined against background solvent optical density (<−0.060):

TABLE 1.1

| | 0 hour | 24 hours | 24 hours | 48 hours | 48 hours |
|---|---|---|---|---|---|
| | (after | (stored at | (stored at | (stored at | (stored at |
| | sonication) | RT) | 5° C.) | RT) | 5° C.) |
| 15% | 0.055 | 0.051 | 0.051 | 0.051 | 0.052 |
| | (n = 3) | (n = 2) | (n = 2) | (pH = 7.28) | (pH = 7.26) |
| 20% | 0.058 | 0.050 | 0.050 | 0.051 | 0.056 |
| | (n = 3) | (n = 2) | (n = 2) | (pH = 7.21) | (pH = 7.28) |

### Example 2—Formulation of TQL-1055 in HP-β-CD solution

Three solutions were prepared: (1) 15% w/v HP-β-CD in 1 ml of PBS, add TQL-1055 (crushed) to achieve 2 mg/ml; (2) 20% w/v HP-β-CD in 1 ml of PBS; add TQL-1055 (crushed) to achieve 2 mg/ml; and (3) 25% w/v HP-β-CD in 1 ml of PBS; add TQL-1055 (crushed) to achieve 2 mg/ml. Solutions were solubilized using heat and sonication for one hour at 60° C. TQL-1055 dissolved completely in both the solutions 2 and 3. 15% (solution 1) was not completely clear (OD >0.060). Optical density measurements shown below:

TABLE 2.1

| | 0 hour (after | 24 hours | 48 hours |
|---|---|---|---|
| | sonication) | (stored at RT) | (stored at RT) |
| 15% | 0.083, 0.083, | 0.081, 0.081 | 0.067, 0.068 |
| | 0.083 (n = 3) | (n = 2) | (n = 2) |
| 20% | 0.061, 0.066, | 0.054, 0.054 | 0.049, 0.053 |
| | 0.068 (n = 3) | (n = 2) | (n = 2) |
| 25% | 0.049, 0.051, | 0.053, 0.054 | 0.057, 0.055 |
| | 0.052 (n = 3) | (n = 2) | (n = 2) |

### Example 3—Stability of TQL-1055 in HP-β-CD solution

Solutions containing 20% w/v HP-β-CD and 1 mg/ml dissolved TQL-1055 were prepared according to the following procedure: (1) crush TQL-1055 and sonicate in PBS; (2) add HP-β-CD solution to create a final concentration of 1 mg/ml TQL-1055, 20% HP-β-CD in PBS buffer (3) sonicate for ˜30-60 minutes at 60C (4) obtain a clear solution having 1 mg/ml TQL-1055; 20% w/v HP-R-CD. Control samples containing 20% HP-β-CD were prepared. Optical density and pH were measured at t=1 day, 1 week, 1 month, and 2 months. HPLC was conducted at t=1 day, 1 week, 1 month, and 2 months. The results are depicted in the tables below.

TABLE 3.1
Clarity (all samples were clear with OD less than 0.06)

| | Temperature | | Day 1 | 1 week | 1 month | 2 month |
|---|---|---|---|---|---|---|
| Vial 1 | −20 | C. | 0.056 | 0.05 | 0.049 | 0.054 |
| Vial 2 | | | 0.052 | 0.051 | 0.0475 | 0.054 |
| Vial 1 | 5 | C. | 0.054 | 0.051 | 0.0485 | 0.058 |
| Vial 2 | | | 0.053 | 0.053 | 0.051 | 0.050 |
| Vial 1 | 25 | C. | 0.050 | 0.052 | 0.0455 | 0.05 |
| Vial 2 | | | 0.052 | 0.049 | 0.053 | 0.051 |
| Control | −20 | C. | 0.048 | 0.051 | 0.051 | 0.055 |
| Control | 5 | C. | 0.049 | 0.054 | 0.048 | 0.051 |
| Control | 25 | C. | 0.047 | 0.057 | 0.055 | 0.052 |

TABLE 3.2
pH

| | Temperature | | Day 1 | 1 week | 1 month | 2 month |
|---|---|---|---|---|---|---|
| Vial 1 | −20 | C. | 7.35 | 7.19 | 7.24 | 7.41 |
| Vial 2 | | | 7.41 | 7.26 | 7.53 | 7.46 |
| Vial 1 | 5 | C. | 7.47 | 7.32 | 7.81 | 7.35 |
| Vial 2 | | | 7.43 | 7.25 | 7.32 | 7.48 |
| Vial 1 | 25 | C. | 7.47 | 7.00 | 7.50 | 7.44 |
| Vial 2 | | | 7.45 | 7.35 | 7.82 | 7.51 |
| Control | −20 | C. | 7.64 | 7.60 | 7.53 | 7.71 |
| Control | 5 | C. | 7.75 | 7.63 | 7.72 | 7.63 |
| Control | 25 | C. | 7.70 | 7.58 | 7.97 | 7.79 |

TABLE 3.3
HPLC

| | Temperature | | Day 1 | 1 week | 1 month | 2 months |
|---|---|---|---|---|---|---|
| Vial 1 | −20 | C. | 100 | 104.7 | 109.6 | 105.3 |
| Vial 2 | | | 105.3 | 116.6* | 109.6 | 102 |
| Vial 1 | 5 | C. | 105.4 | 104.4 | 106.1 | 109 |
| Vial 2 | | | 109.4 | 108.9 | 107.9 | 105 |
| Vial 1 | 25 | C. | 109.4 | 106.9 | 107.5 | 105 |
| Vial 2 | | | 106.1 | 109.2 | 108.7 | 105.0 |
| Control | −20 | C. | | | | |
| Control | 5 | C. | | | | |
| Control | 25 | C. | | | | |

*outliers are attributed to sample preparation variation and method variation. The method used is still being validated.

### Example 4—Immunogenicity of TQL-1055 in HP-β-CD solution

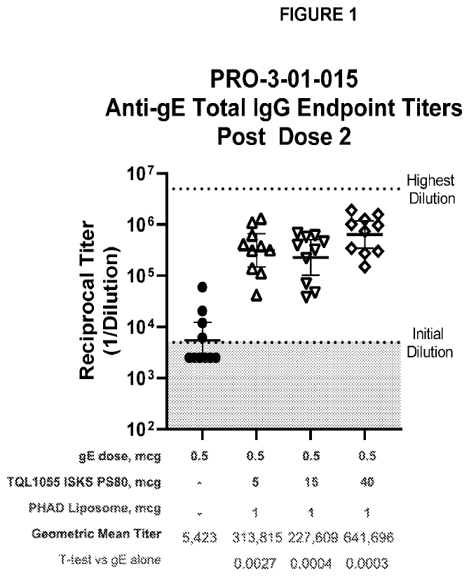

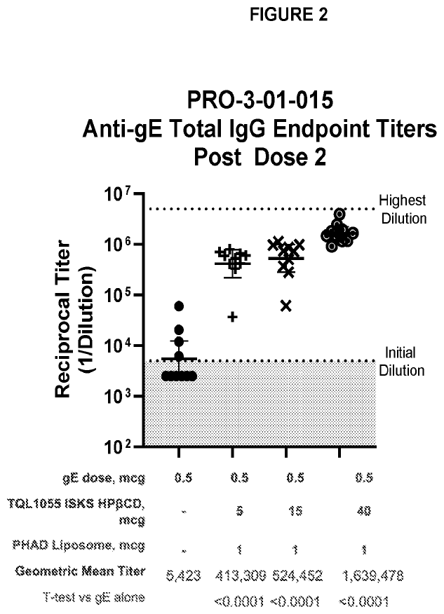

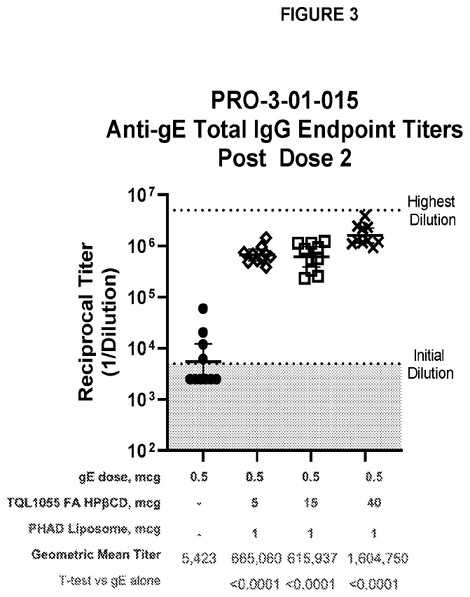

Mice were immunized on day 0 and day 14 with formulations having TQL-1055 (in situ potassium (carbonate) salt or free acid), Varicella Zoster gE, PHAD (TLR4 agonist) in DOPC/cholesterol liposomes, and 3.75% w/v polysorbate 80 (PS80) or 10/20% w/v HP-β-CD. Mice were bled on day 28 to measure anti-gE total IgG endpoint titers across all groups. The results are shown in FIGS. 1 to 3. In these figures, “ISKS” formulations contain in situ potassium (carbonate) salt—10 molar equivalents of K₂CO₃ to TQL-1055. ISKS PS80 formulations contain 3.75% PS80 in PBS at 1 mg/ml TQL-1055; ISKS HP-β-CD (HPβCD) formulations contain 10% w/v HP-β-CD in PBS and 1 mg/ml TQL-1055. FA HP-β-CD formulations contain 20% w/v HP-β-CD in PBS with 1 mg/ml TQL-1055. The data demonstrate HP-T3-CD formulations are immunogenic and outperform PS80 formulations.

### Example 5—Tolerability of TQL-1055 in HP-R-CD solution

The tolerability of soluble TQL-1055 formulations, including TQL-1055 FA (free acid) HP-β-CD and TQL-1055 ISKS (in situ potassium salt) HP-β-CD formulations, administered over a range of doses was examined across three mouse strains (C57BL/6J, BALB/cJ, CD-i). The following experimental groups were used. I.M. administration was performed.

| | | | | TQL1055 | TQL1055 | |
|---|---|---|---|---|---|---|
| | C57BL/ | BALB/ | CD- | FA 20% | ISKS 10% | |
| Grp | 6J | c | 1 | HPβCD | HPβCD | QS-21 |
| 1 | 10 | 10 | 10 | 20 μg | — | — |
| 2 | 10 | 10 | 10 | 40 μg | — | — |
| 3 | 10 | 10 | 10 | 60 μg | — | — |
| 4 | 10 | 10 | 10 | 80 μg | — | — |
| 5 | 10 | 10 | 10 | 100 μg | — | — |
| 6 | 10 | 10 | 10 | — | 20 μg | — |
| 7 | 10 | 10 | 10 | — | 40 μg | — |
| 8 | 10 | 10 | 10 | — | 60 μg | — |
| 9 | 10 | 10 | 10 | — | 80 μg | — |
| 10 | 10 | 10 | 10 | — | 100 μg | — |
| 11 | 10 | 10 | 10 | — | — | 20 μg |
| 12 | 10 | 10 | 10 | — | — | 40 μg |
| 13 | 10 | 10 | 10 | — | — | 60 μg |
| 14 | 10 | 10 | 10 | — | — | 80 μg |
| 15 | 10 | 10 | 10 | — | — | 100 μg |

The mortality rate 7 days after injection was observed. The following table shows the number of deaths in each group for each formulation. As indicated below, none of the mice receiving TQL-1055 ISKS HPβCD died, whereas at least some mice in the other groups (including QS-21) died.

| Treatment | C57BL/6J | BALB/cJ | CD-1 |
|---|---|---|---|
| Groups | Deaths | Deaths | Deaths |
| 20 μg TQL1055 FA | 0/10 | 0/10 | 0/10 |
| HPβCD | | | |
| 40 μg TQL1055 FA | 0/10 | 0/10 | 0/10 |
| HPβCD | | | |
| 60 μg TQL1055 FA | 0/10 | 0/10 | 3/10 |
| HPβCD | | | |
| 80 μg TQL1055 FA | 1/10 | 5/10 | 7/10 |
| HPβCD | | | |
| 100 μg TQL1055 | 7/10 | 5/10 | 3/10 |
| FA HPβCD | | | |
| 20 μg TQL1055 | 0/10 | 0/10 | 0/10 |
| ISKS HPβCD | | | |
| 40 μg TQL1055 | 0/10 | 0/10 | 0/10 |
| ISKS HPβCD | | | |
| 60 μg TQL1055 | 0/10 | 0/10 | 0/10 |
| ISKS HPβCD | | | |
| 80 μg TQL1055 | 0/10 | 0/10 | 0/10 |
| ISKS HPβCD | | | |
| 100 μg TQL1055 | 0/10 | 0/10 | 0/10 |
| ISKS HPβCD | | | |
| 20 μg QS-21 | 0/10 | 0/10 | 0/10 |
| 40 μg QS-21 | 2/10 | 0/10 | 0/10 |
| 60 μg QS-21 | 0/10 | 0/10 | 0/10 |
| 80 μg QS-21 | 5/10 | 0/10 | 0/10 |
| 100 μg QS-21 | 10/10 | 0/10 | 0/10 |

A Logrank (Mantel-Cox) test was performed to assess survival distributions of the foregoing groups. The comparison results are shown below.

| Comparison | | |
|---|---|---|
| Groups (C57BL/6 mice) | Dose | P value |
| TQL1055 FA HPβCD Vs. | 20 μg | >0.9999 |
| TQL1055 ISKS HPβCD | 40 μg | >0.9999 |
| | 60 μg | >0.9999 |
| | 80 μg | 0.3173 |
| | 100 μg | 0.0013 |

| Comparison | | |
|---|---|---|
| Groups (C57BL/6 mice) | Dose | P value |
| TQL1055 FA HPβCD Vs. | 20 μg | >0.9999 |
| QS-21 | 40 μg | 0.1672 |
| | 60 μg | >0.9999 |
| | 80 μg | 0.0826 |
| | 100 μg | 0.7941 |

| Comparison | | |
|---|---|---|
| Groups (C57BL/6 mice) | Dose | P value |
| TQL1055 ISKS HPβCD Vs. | 20 μg | >0.9999 |
| QS-21 | 40 μg | 0.1672 |
| | 60 μg | >0.9999 |
| | 80 μg | 0.012 |
| | 100 μg | <0.0001 |

| Comparison | | |
|---|---|---|
| Groups (BALB/c mice) | Dose | P value |
| TQL1055 FA HPβCD Vs. | 20 μg | >0.9999 |
| TQL1055 ISKS HPβCD | 40 μg | >0.9999 |
| | 60 μg | >0.9999 |
| | 80 μg | 0.0115 |
| | 100 μg | 0.0116 |

| Comparison | | |
|---|---|---|
| Groups (BALB/c mice) | Dose | P value |
| TQL1055 FA HPβCD Vs. | 20 μg | >0.9999 |
| QS-21 | 40 μg | >0.9999 |
| | 60 μg | >0.9999 |
| | 80 μg | 0.0115 |
| | 100 μg | 0.0116 |

| Comparison | | |
|---|---|---|
| Groups (BALB/c mice) | Dose | P value |
| TQL1055 ISKS HPβCD Vs. | 20 μg | >0.9999 |
| QS-21 | 40 μg | >0.9999 |
| | 60 μg | >0.9999 |
| | 80 μg | >0.9999 |
| | 100 μg | >0.9999 |

| Comparison | | |
|---|---|---|
| Groups (CD-1 mice) | Dose | P value |
| TQL1055 FA HPβCD Vs. | 20 μg | >0.9999 |
| TQL1055 ISKS HPβCD | 40 μg | >0.9999 |
| | 60 μg | 0.0671 |
| | 80 μg | 0.0024 |
| | 100 μg | 0.0671 |

| Comparison | | |
|---|---|---|
| Groups (CD-1 mice) | Dose | P value |
| TQL1055 FA HPβCD Vs. | 20 μg | >0.9999 |
| QS-21 | 40 μg | >0.9999 |
| | 60 μg | 0.0671 |
| | 80 μg | 0.0024 |
| | 100 μg | 0.0671 |

Comparison

| Groups (CD-1 mice) | Dose | P value |
|---|---|---|
| TQL1055 ISKS HPβCD Vs. QS-21 | 20 μg | >0.9999 |
| | 40 μg | >0.9999 |
| | 60 μg | >0.9999 |
| | 80 μg | >0.9999 |
| | 100 μg | >0.9999 |

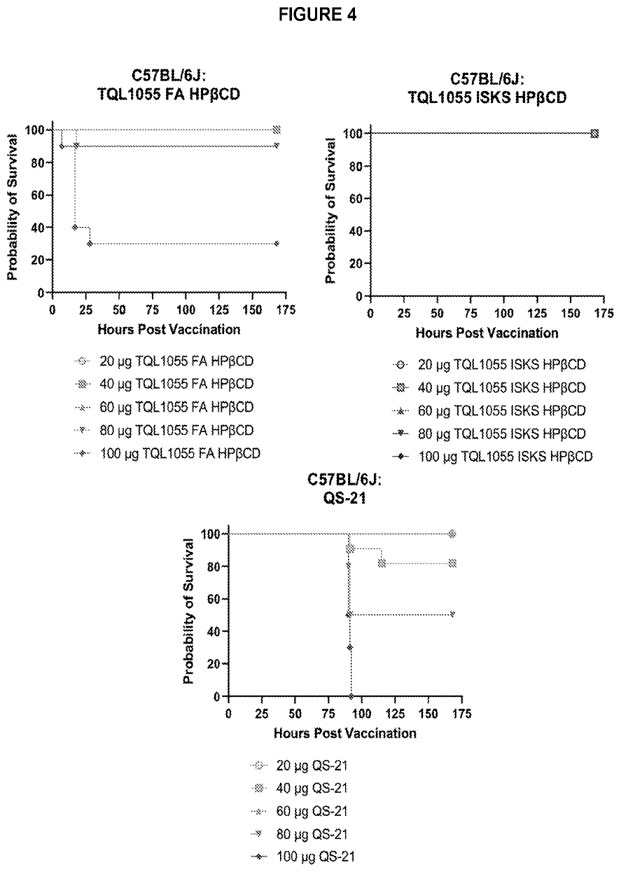

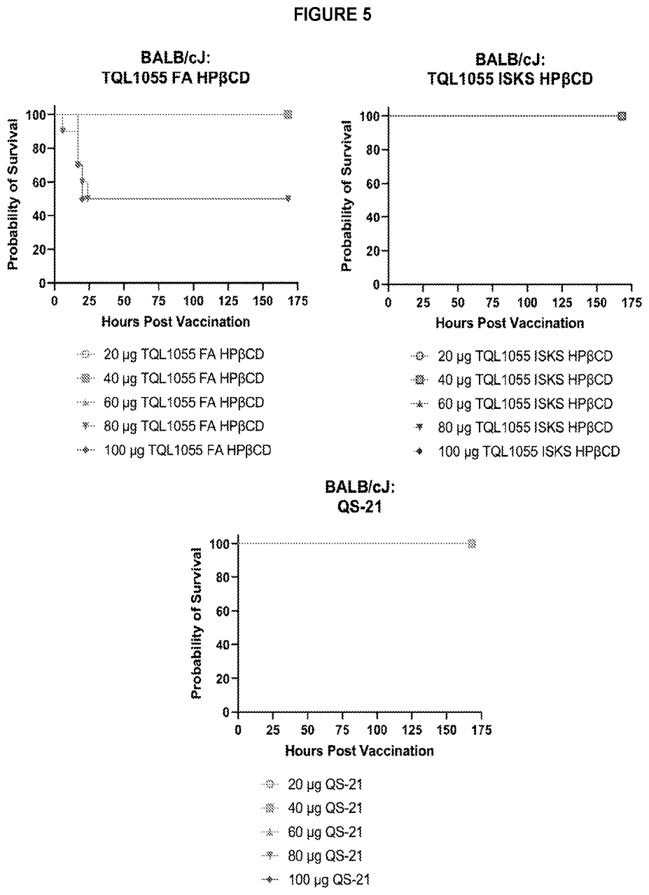

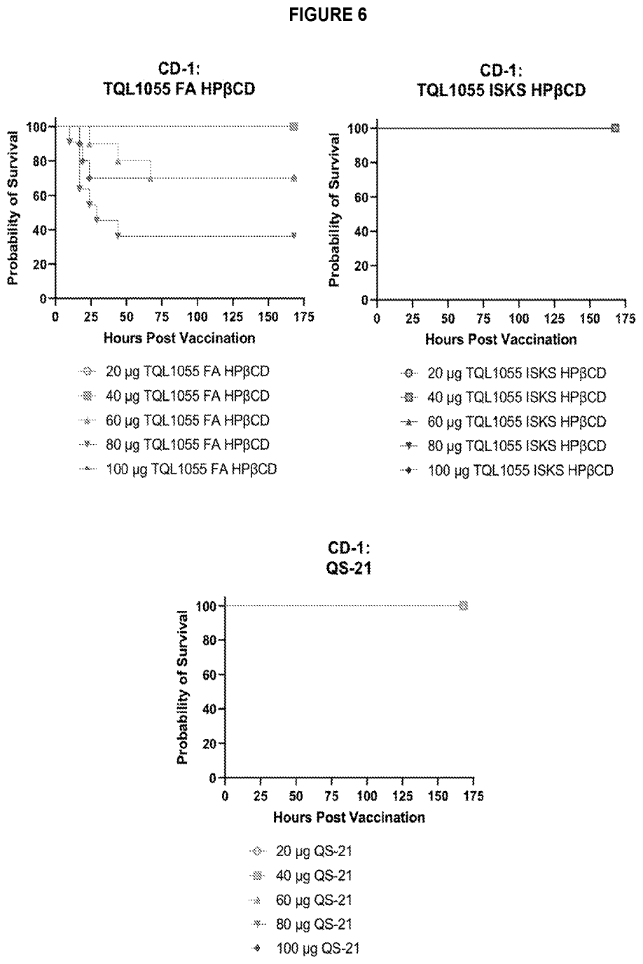

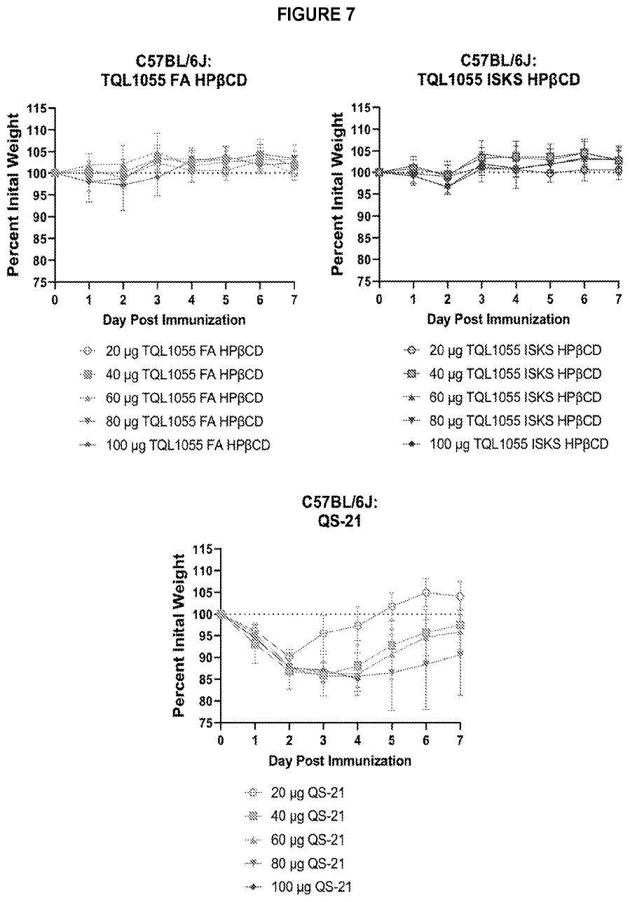

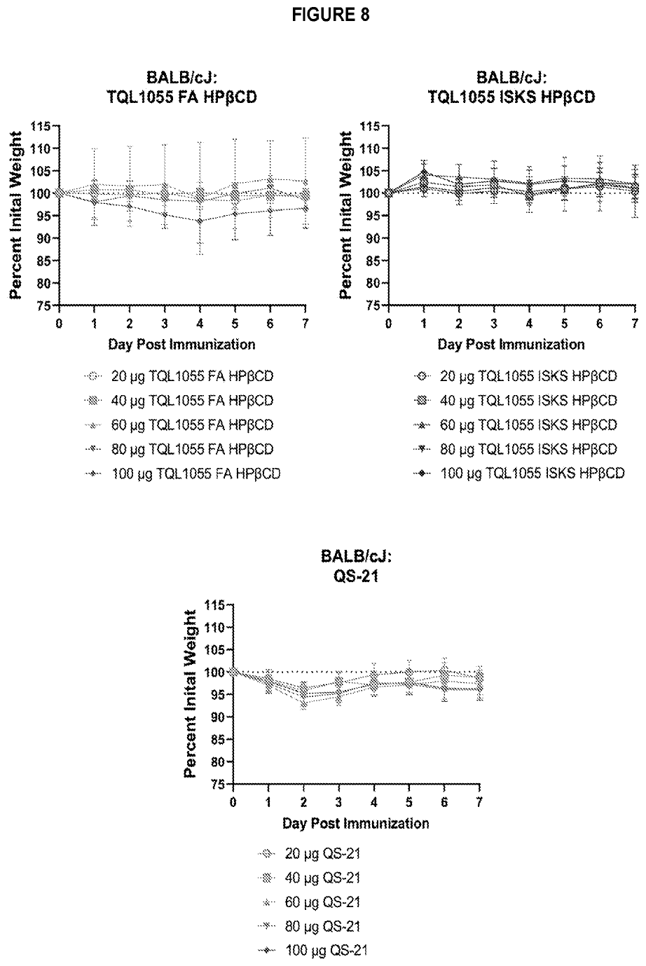

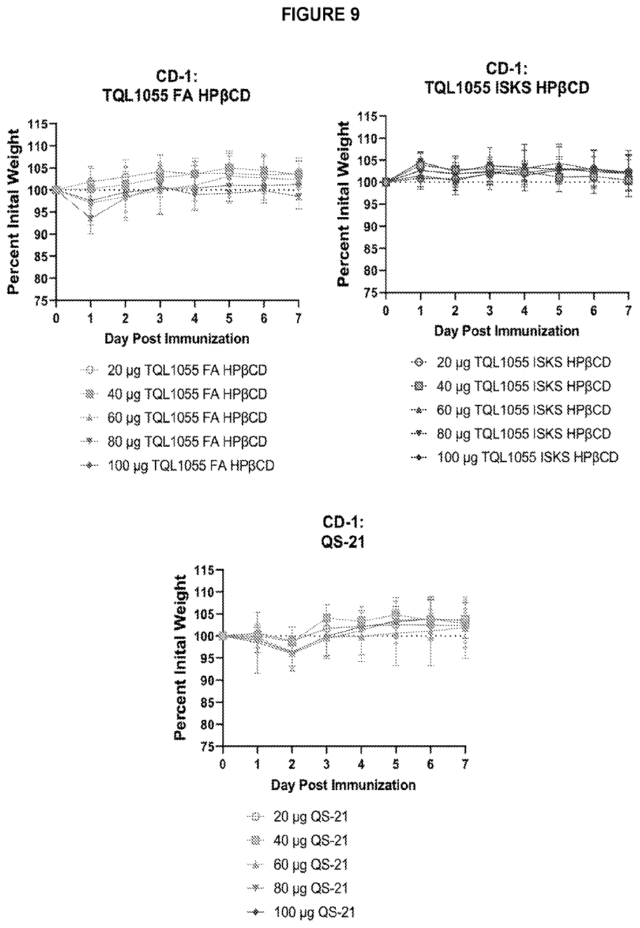

FIGS. 4 to 6 depict the survival rates for each formulation for each mouse strain. FIGS. 7 to 9 depict weight loss data observed for each mouse strain.

No mortality was observed in any mouse strain with TQL-1055 ISKS HPβCD. Tolerability to TQL1055 FA HPβCD and TQL1 055 ISKS HPβCD appear to differ at high doses. TQL1055 FA HPβCD results in statistically significantly more death at higher doses (80 and/or 100 μg) than TQL1055 ISKS HPβCD in all mouse strains. Mouse strain appears to play a role in both TQL1055 FA HPβCD and QS-21 tolerability, in particular, CD-i mice may be more sensitive to TQL1 055 FA HPβCD than other strains, and C57BL/6 mice appear to be more sensitive to QS-21 than other strains.

### Example 6—TQL-1055 HP-Q-CD Dose Ranging Study

A study was performed to examine the tolerability and antibody response of soluble TQL-1 055 formulations (free acid (FA) hydroxypropyl-P3-cyclodextrin (HPβCD) and in situ potassium salt (ISKS) HPβCD) administered over a range of doses using an influenza (A/Puerto Rico/8/1934, PR8) hemagglutinin (HA) vaccination model compared to TQL1055 FA in a micro suspension. The study also compared tolerability of TQL-1055 formulations to QS-21. The antigen used was PR8 HA (0.1 mcg). The Adjuvants used were TQL1055 FA 20% HβCD, TQL1055 ISKS 10% HβCD, TQL1055 FA, HPβCD alone, and QS-21. Route of administration was I.M. CD-i mice were vaccinated on Day 0 and Day 14. Mice were bled on Day 28 to assess total anti-HA total IgG endpoint titers. The vaccination groups are shown in the table below:

| | | PR8 | | | | | |
|---|---|---|---|---|---|---|---|
| | CD- | HA | TQL1055 | TQL1055 | | | |
| | 1 | Anti- | FA | ISKS | TQL1055 | HPβCD | QS- |
| Grp | Mice | gen | HPβCD | HPβCD | FA | Alone | 21 |
| 1 | 10 | 0.1 μg | — | — | — | — | — |
| 2 | 10 | 0.1 μg | 50 μg | — | — | — | — |
| 3 | 10 | 0.1 μg | 100 μg | — | — | — | — |
| 4 | 10 | 0.1 μg | 198.68 μg | — | — | — | — |
| 5 | 10 | 0.1 μg | — | 50 μg | — | — | — |
| 6 | 10 | 0.1 μg | — | 100 μg | — | — | — |
| 7 | 10 | 0.1 μg | — | 198.68 μg | — | — | — |
| 8 | 10 | 0.1 μg | — | — | 50 μg | — | — |
| 9 | 10 | 0.1 μg | — | — | 100 μg | — | — |
| 10 | 10 | 0.1 μg | — | — | 198.68 μg | — | — |
| 11 | 10 | 0.1 μg | — | — | — | Dose | — |
| | | | | | | equiv- | |
| | | | | | | alent | |
| | | | | | | to | |
| | | | | | | group | |
| | | | | | | 4 | |
| 12 | 10 | 0.1 μg | — | — | — | — | 25 μg |
| 13 | 10 | 0.1 μg | — | — | — | — | 50 μg |
| 14 | 10 | 0.1 μg | — | — | — | — | 100 μg |
| 15 | 10 | — | 50 μg | — | — | — | — |
| 16 | 10 | — | 100 μg | — | — | — | — |
| 17 | 10 | — | 200 μg | — | — | — | — |
| 18 | 10 | — | — | 50 μg | — | — | — |
| 19 | 10 | — | — | 100 μg | — | — | — |
| 20 | 10 | — | — | 200 μg | — | — | — |
| 21 | 10 | — | — | — | 50 μg | — | — |
| 22 | 10 | — | — | — | 100 μg | — | — |
| 23 | 10 | — | — | — | 200 μg | — | — |
| 24 | 10 | — | — | — | — | Dose | |
| | | | | | | equiv- | |
| | | | | | | alent | |
| | | | | | | to | |
| | | | | | | group | |
| | | | | | | 4 | |

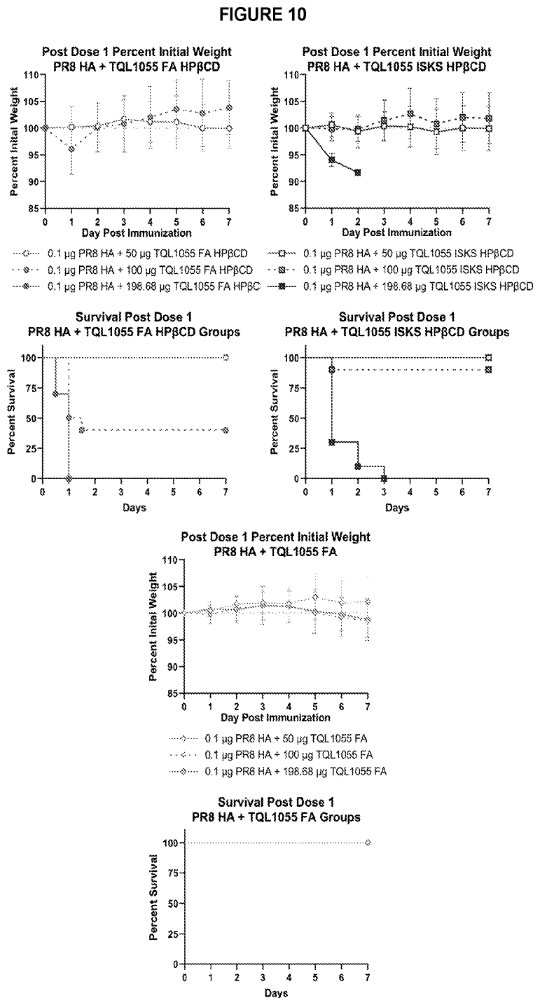

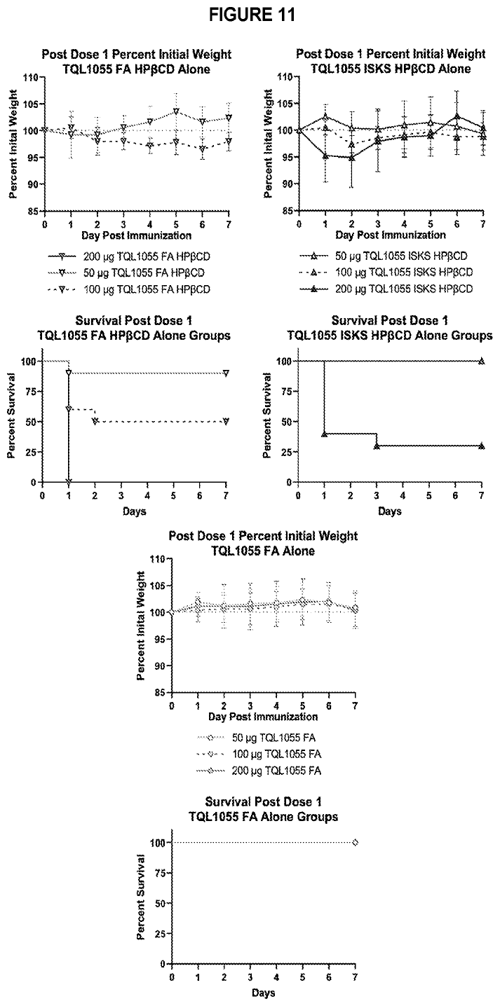

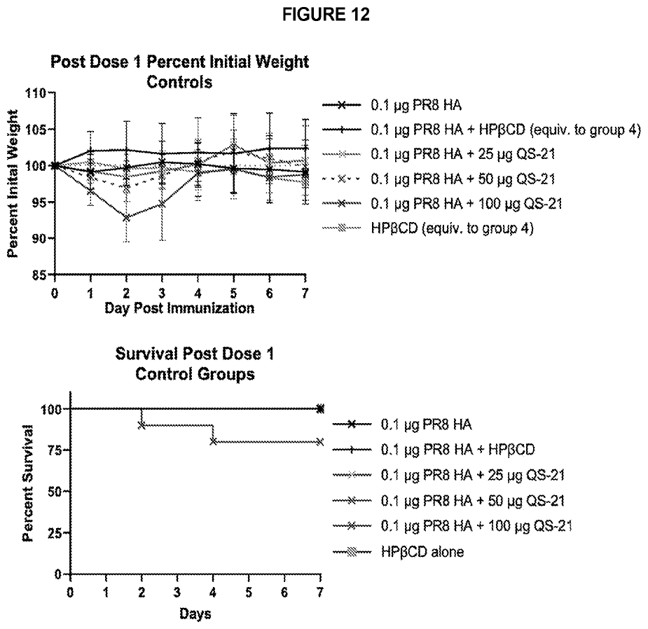

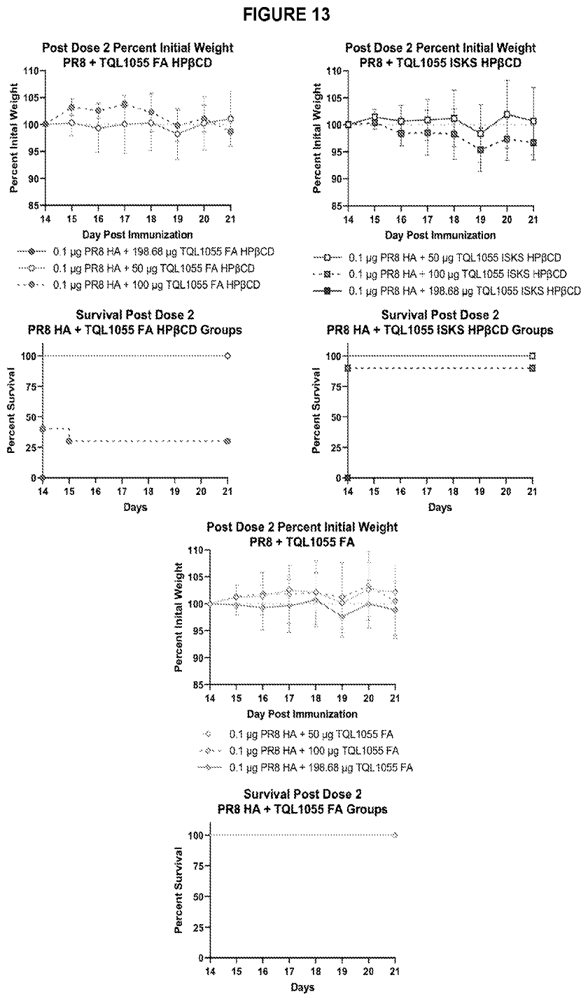

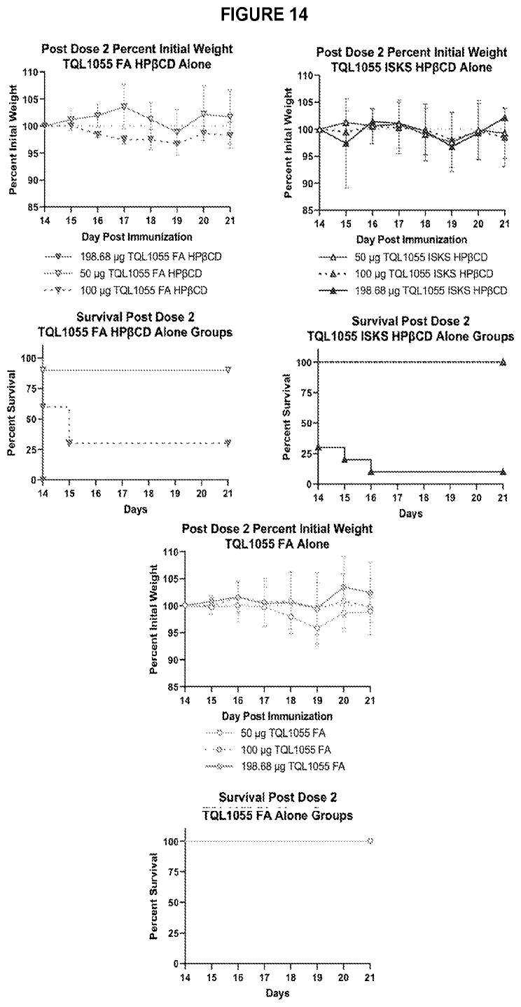

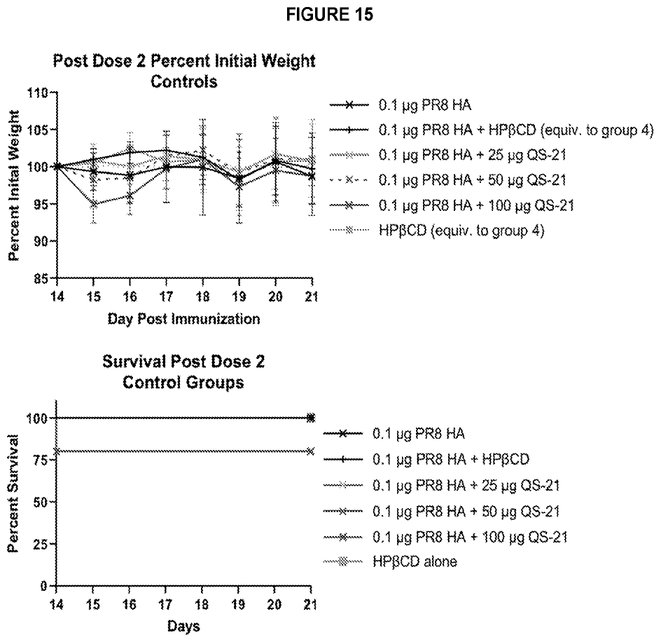

FIGS. 10 to 12 depict weight loss and survival data post dose 1 (administered on Day 0). FIGS. 13 to 15 depict weight loss and survival data post dose 2 (administered on day 14).

A summary table of weight loss on Day 2 and Survival on Day 7 appears below:

| | Mean initial | Deaths |
|---|---|---|
| Treatment Groups | weight (D 2) | (D 7) |
| 0.1 μg PR8 HA | 99.72 | 0/10 |
| 0.1 μg PR8 HA + 50 μg TQL1055 FA HPβCD | 100.40 | 0/10 |
| 0.1 μg PR8 HA + 100 μg TQL1055 FA HPβCD | 100.11 | 6/10 |
| 0.1 μg PR8 HA + 198.68 μg TQL1055 FA | NA | 10/10 |
| HPβCD | | |
| 0.1 μg PR8 HA + 50 μg TQL1055 ISKS HPβCD | 99.36 | 0/10 |
| 0.1 μg PR8 HA + 100 μg TQL1055 ISKS HPβCD | 99.77 | 1/10 |
| 0.1 μg PR8 HA + 198.68 μg TQL1055 ISKS | 91.67 | 10/10 |
| HPβCD | | |
| 0.1 μg PR8 HA + 50 μg TQL1055 FA | 101.65 | 0/10 |
| 0.1 μg PR8 HA + 100 μg TQL1055 FA | 100.93 | 0/10 |
| 0.1 μg PR8 HA + 198.68 μg TQL1055 FA | 100.61 | 0/10 |
| 0.1 μg PR8 HA + HPβCD alone | 102.15 | 0/10 |
| 0.1 μg PR8 HA + 25 μg QS-21 | 98.45 | 0/10 |
| 0.1 μg PR8 HA + 50 μg QS-21 | 96.94 | 0/10 |
| 0.1 μg PR8 HA + 100 μg QS-21 | 92.87 | 2/10 |
| 50 μg TQL1055 FA HPβCD | 99.20 | 1/10 |
| 100 μg TQL1055 FA HPβCD | 97.96 | 5/10 |
| 200 μg TQL1055 FA HPβCD | NA | 10/10 |
| 50 μg TQL1055 ISKS HPβCD | 100.37 | 0/10 |
| 100 μg TQL1055 ISKS HPβCD | 97.37 | 0/10 |
| 200 μg TQL1055 ISKS HPβCD | 94.86 | 7/10 |
| 50 μg TQL1055 FA | 101.23 | 0/10 |
| 100 μg TQL1055 FA | 100.62 | 0/10 |
| 200 μg TQL1055 FA | 101.12 | 0/10 |
| HPβCD alone | 99.58 | 0/10 |

A Logrank (Mantel-Cox) test was performed to assess survival distributions of the foregoing groups. The comparison results are shown below.

| Survival by Log-rank (Mantel-Cox) test (post dose 1) | | | P value |
|---|---|---|---|
| PR8 HA + TQL1055 FA HPβCD Vs. | 100 | μg | 0.0222 |
| PR8 HA + TQL1055 ISKS HPβCD | 198.68 | μg | 0.0167 |
| PR8 HA + TQL1055 FA HPβCD Vs. | 100 | μg | 0.0040 |
| PR8 HA + TQL1055 FA | 198.68 | μg | <0.0001 |
| PR8 HA + TQL1055 ISKS HPβCD Vs. | 100 | μg | 0.3173 |
| PR8 HA + TQL1055 FA | 198.68 | μg | <0.0001 |
| PR8 HA + TQL1055 FA HPβCD Vs. | 100 | μg | 0.0436 |
| PR8 HA + QS-21 | | | |
| PR8 HA + TQL1055 ISKS HPβCD Vs. | 100 | μg | 0.5842 |
| PR8 HA + QS-21 | | | |
| PR8 HA + TQL1055 FA Vs. | 100 | μg | 0.1464 |
| PR8 HA + QS-21 | | | |
| TQL1055 FA HPβCD Vs. | 100 | μg | 0.0114 |
| TQL1055 ISKS HPβCD | 200 | μg | 0.0293 |
| TQL1055 FA HPβCD Vs. | 100 | μg | 0.0114 |
| TQL1055 FA | 200 | μg | <0.0001 |
| TQL1055 ISKS HPβCD Vs. | 100 | μg | >0.9999 |
| TQL1055 FA | 200 | μg | 0.0012 |

A summary table of weight loss on Day 15 and Survival on Day 21 appears below:

| | Mean initial | |
|---|---|---|
| | weight | Deaths |
| Treatment Groups | (D 15) | (D 21) |
| 0.1 μg PR8 HA | 99.34 | 0/10 |
| 0.1 μg PR8 HA + 50 μg TQL1055 FA HPβCD | 100.31 | 0/10 |
| 0.1 μg PR8 HA + 100 μg TQL1055 FA HPβCD | 103.21 | 1/4 |
| 0.1 μg PR8 HA + 198.68 μg TQL1055 FA | N/A | N/A |
| HPβCD | | |
| 0.1 μg PR8 HA + 50 μg TQL1055 ISKS HPβCD | 101.44 | 0/10 |
| 0.1 μg PR8 HA + 100 μg TQL1055 ISKS HPβCD | 100.39 | 0/9 |
| 0.1 μg PR8 HA + 198.68 μg TQL1055 ISKS | N/A | N/A |
| HPβCD | | |
| 0.1 μg PR8 HA + 50 μg TQL1055 FA | 101.18 | 0/10 |
| 0.1 μg PR8 HA + 100 μg TQL1055 FA | 101.31 | 0/10 |
| 0.1 μg PR8 HA + 198.68 μg TQL1055 FA | 99.76 | 0/10 |
| 0.1 μg PR8 HA + HPβCD alone | 100.98 | 0/10 |
| 0.1 μg PR8 HA + 25 μg QS-21 | 100.82 | 0/10 |
| 0.1 μg PR8 HA + 50 μg QS-21 | 98.21 | 0/10 |
| 0.1 μg PR8 HA + 100 μg QS-21 | 94.93 | 0/8 |
| 50 μg TQL1055 FA HPβCD | 101.17 | 0/9 |
| 100 μg TQL1055 FA HPβCD | 100.00 | 3/6 |
| 200 μg TQL1055 FA HPβCD | N/A | N/A |
| 50 μg TQL1055 ISKS HPβCD | 101.23 | 0/10 |
| 100 μg TQL1055 ISKS HPβCD | 99.44 | 0/10 |
| 200 μg TQL1055 ISKS HPβCD | 97.39 | 2/3 |
| 50 μg TQL1055 FA | 99.65 | 0/10 |
| 100 μg TQL1055 FA | 99.92 | 0/10 |
| 200 μg TQL1055 FA | 100.79 | 0/10 |
| HPβCD alone | 100.46 | 0/10 |

A Logrank (Mantel-Cox) test was performed to assess survival distributions of the foregoing groups. The comparison results are shown below.

| Survival by Log-rank (Mantel-Cox) test | | | P value |
|---|---|---|---|
| PR8 HA + TQL1055 FA HPβCD Vs. | 100 | μg | 0.0072 |
| PR8 HA + TQL1055 ISKS HPβCD | 198.68 | μg | >0.9999 |
| PR8 HA + TQL1055 FA HPβCD Vs. | 100 | μg | 0.0012 |
| PR8 HA + TQL1055 FA | 198.68 | μg | <0.0001 |
| PR8 HA + TQL1055 ISKS HPβCD Vs. | 100 | μg | 0.3173 |
| PR8 HA + TQL1055 FA | 198.68 | μg | <0.0001 |
| PR8 HA + TQL1055 FA HPβCD Vs. | 100 | μg | 0.0287 |
| PR8 HA + QS-21 | | | |
| PR8 HA + TQL1055 ISKS HPβCD Vs. | 100 | μg | 0.5416 |
| PR8 HA + QS-21 | | | |
| PR8 HA + TQL1055 FA Vs. | 100 | μg | 0.1462 |
| PR8 HA + QS-21 | | | |
| TQL1055 FA HPβCD Vs. | 100 | μg | 0.0013 |
| TQL1055 ISKS HPβCD | 200 | μg | 0.0671 |
| TQL1055 FA HPβCD Vs. | 100 | μg | 0.0013 |
| TQL1055 FA | 200 | μg | <0.0001 |
| TQL1055 ISKS HPβCD Vs. | 100 | μg | >0.9999 |
| TQL1055 FA | 200 | μg | <0.0001 |

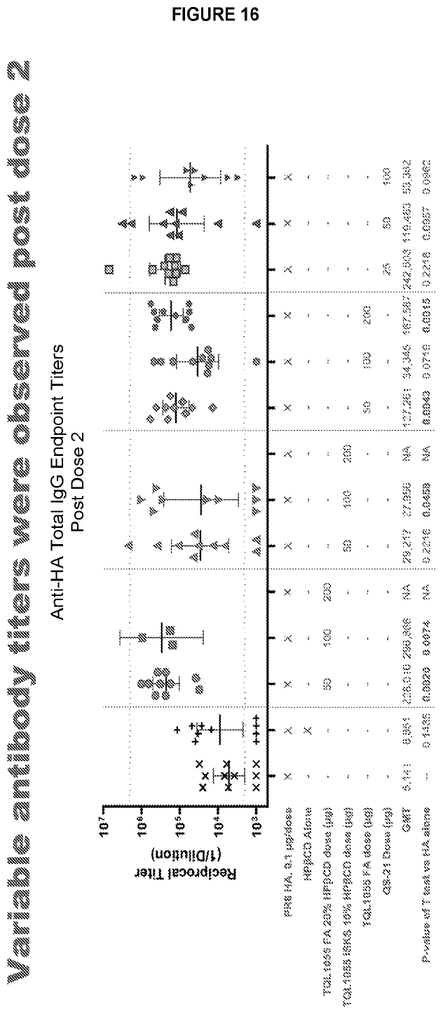

FIG. 16 depicts anti-HA total IgG endpoint titers post dose 2 for each group.

### Example 7—Comparisons of the adjuvanticity of TQL-1055 HP-β-CD formulations with and without liposomal EcML in a VZV gE antigen vaccine

A study was performed to compare the adjuvanticity of three TQL1055 hydroxypropyl p cyclodextrin (HP p CD) formulations (1 mg/mL TQL1055 FA with 20% HP p CD, 2 mg/mL TQL1055 FA with 20% HP p CD, and 2 mg/mL TQL1055 in situ potassium salt (ISKS) with 10% HP p CD) using a fixed dose of varicella zoster virus (VZV) gE. The study also compared the adjuvanticity of the three TQL1055 HP p CD formulations in combination with a fixed dose of EcML containing liposomes and a fixed dose of gE. The study further investigated the antibody responses induced by gE adjuvanted with each TQL1055 HP-β-CD formulation in combination with EcML containing liposomes that were mixed 6 hours before administration.

The antigen used was VZV gE (0.5 μg). The adjuvants used were TQL1055 FA HPβCD (20%) (1 mg/mL) (15 or 40 μg), TQL1055 FA HPβCD (20%) (2 mg/mL) (15, 40, or 80 μg), TQL1055 ISKS HPβCD (10%) (2 mg/mL) (15, 40, or 80 μg), and EcML Liposomes (1 μg). Route of administration was I.M on DO and D14. Antigen-specific ELISA for anti-gE total IgG (D13, D28, D42) was performed. The treatment groups are shown in the table below:

| | | | | TQL1055 | TQL1055 | TQL1055 |
|---|---|---|---|---|---|---|
| | | | | FA 20% | FA 20% | ISKS 10% |
| | CD-1 | | | HPβCD | HPβCD | HPβCD |
| Group | Mice | gE | EcML | (1 mg/mL) | (2 mg/mL) | (2 mg/mL) |
| 1 | 10 | 0.5 μg | — | — | — | — |
| 2 | 10 | 0.5 μg | 1 μg | — | — | — |
| 3 | 10 | 0.5 μg | — | 15 μg | — | — |
| 4 | 10 | 0.5 μg | — | 40 μg | — | — |
| 5 | 10 | 0.5 μg | — | — | 15 μg | — |
| 6 | 10 | 0.5 μg | — | — | 40 μg | — |
| 7 | 10 | 0.5 μg | — | — | 80 μg | — |
| 8 | 10 | 0.5 μg | — | — | — | — |
| 9 | 10 | 0.5 μg | — | — | — | — |
| 10 | 10 | 0.5 μg | — | — | — | — |
| 11 | 10 | 0.5 μg | 1 μg | 15 μg | — | — |
| 12 | 10 | 0.5 μg | 1 μg | 40 μg | — | — |
| 13 | 10 | 0.5 μg | 1 μg | 40 μg | — | — |
| | | | | (6 h admix) | | — |
| 14 | 10 | 0.5 μg | 1 μg | — | 15 μg | — |
| 15 | 10 | 0.5 μg | 1 μg | — | 40 μg | — |
| 16 | 10 | 0.5 μg | 1 μg | — | 40 μg | — |
| | | | | | (6 h admix) | |
| 17 | 10 | 0.5 μg | 1 μg | — | 80 μg | — |
| 18 | 10 | 0.5 μg | 1 μg | — | 80 μg | |
| | | | | | (6 h admix) | |
| 19 | 10 | 0.5 μg | 1 μg | — | — | 15 μg |
| 20 | 10 | 0.5 μg | 1 μg | — | — | 40 μg |
| 21 | 10 | 0.5 μg | 1 μg | — | — | 40 μg |
| | | | | | | (6 h admix) |
| 22 | 10 | 0.5 μg | 1 μg | — | — | 80 μg |
| 23 | 10 | 0.5 μg | 1 μg | — | — | 80 μg |
| | | | | | | (6 h admix) |

Animal deaths in the study are shown below:

| Group | Deaths |
|---|---|
| 1 | — |
| 2 | — |
| 3 | — |
| 4 | — |
| 5 | — |
| 6 | — |
| 7 | 3 (found dead) |
| 8 | — |
| 9 | — |
| 10 | — |
| 11 | 1 (euthanized due to suspected ear infection) |
| 12 | — |
| 13 | — |
| 14 | — |
| 15 | — |
| 16 | — |
| 17 | 4 (found dead) |
| 18 | — |
| 19 | — |
| 20 | — |
| 21 | — |
| 22 | — |
| 23 | — |

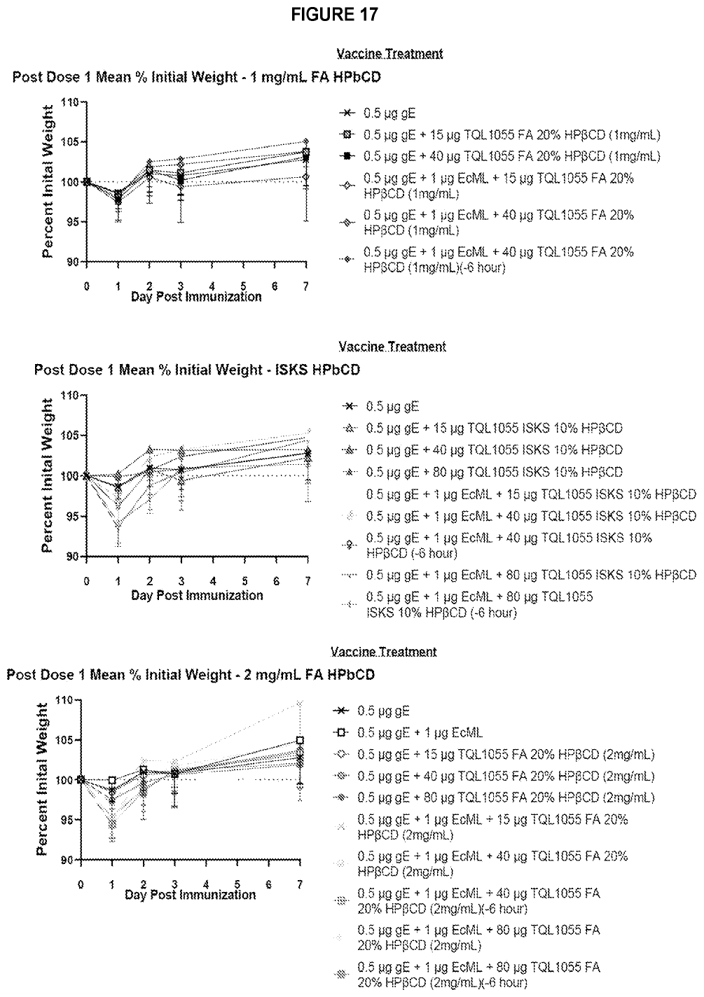

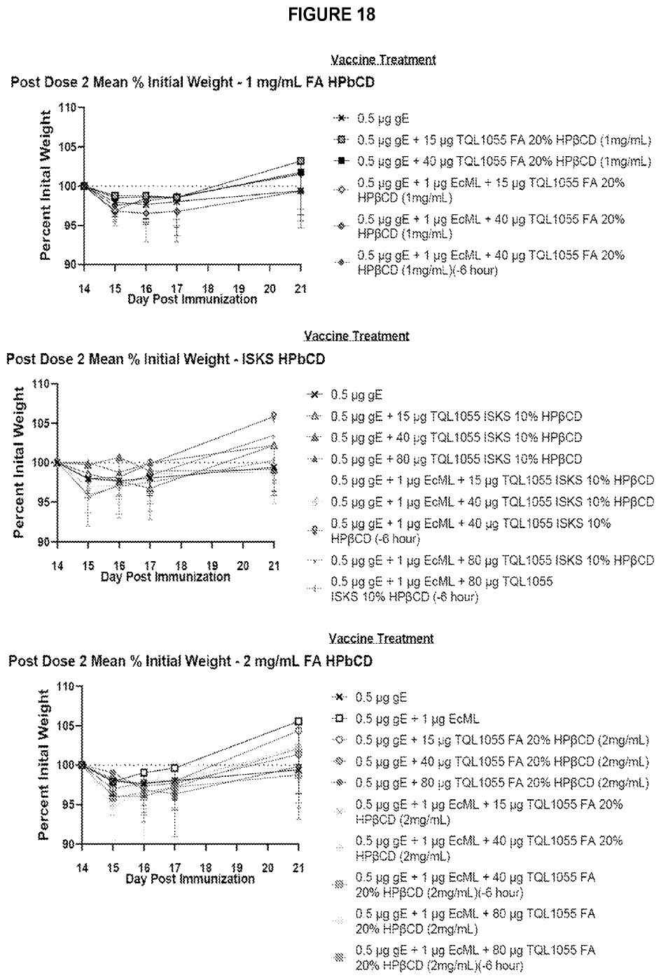

FIG. 17 depicts animal weight data after dose 1. FIG. 18 depicts animal weight after dose 2.

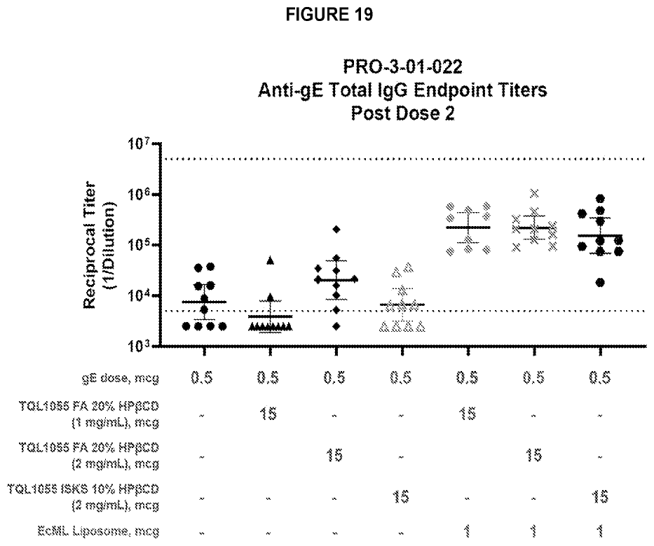

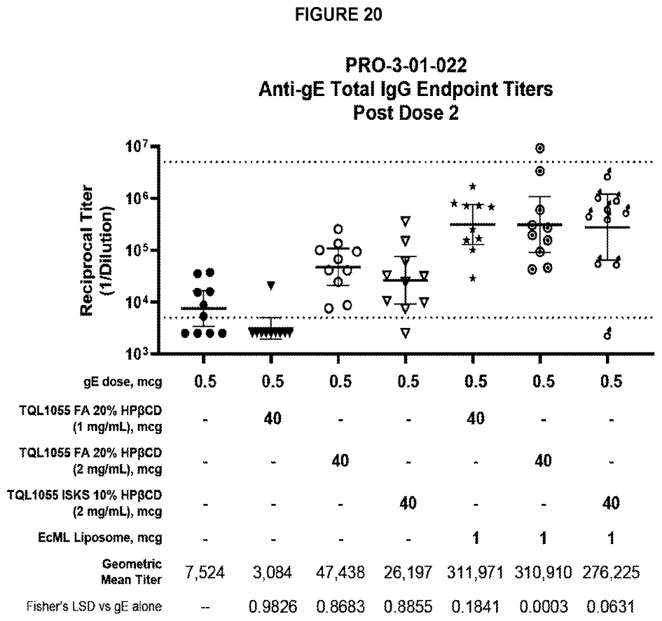

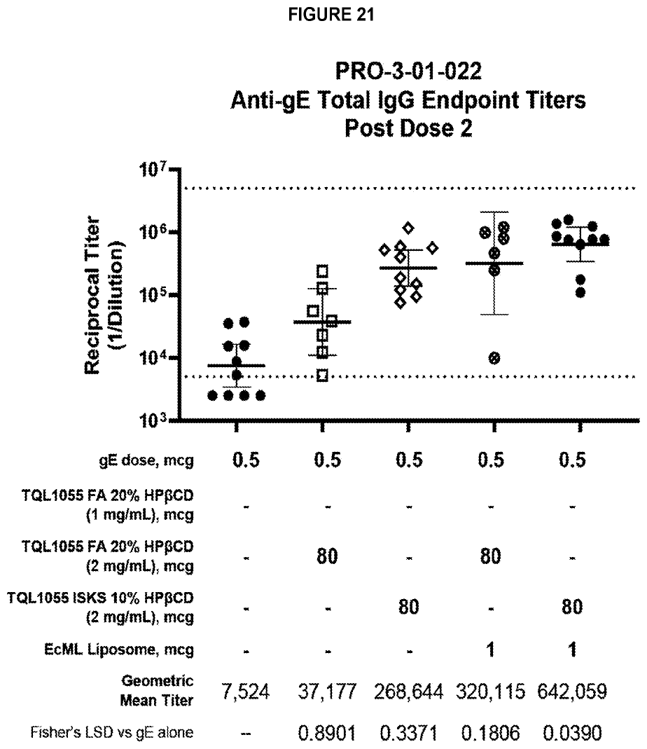

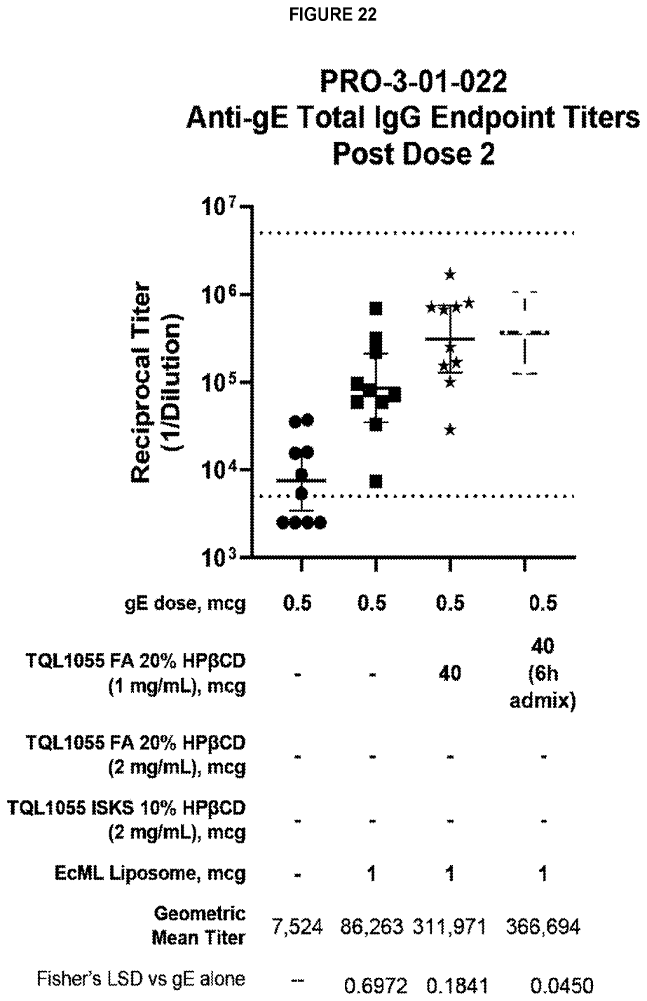

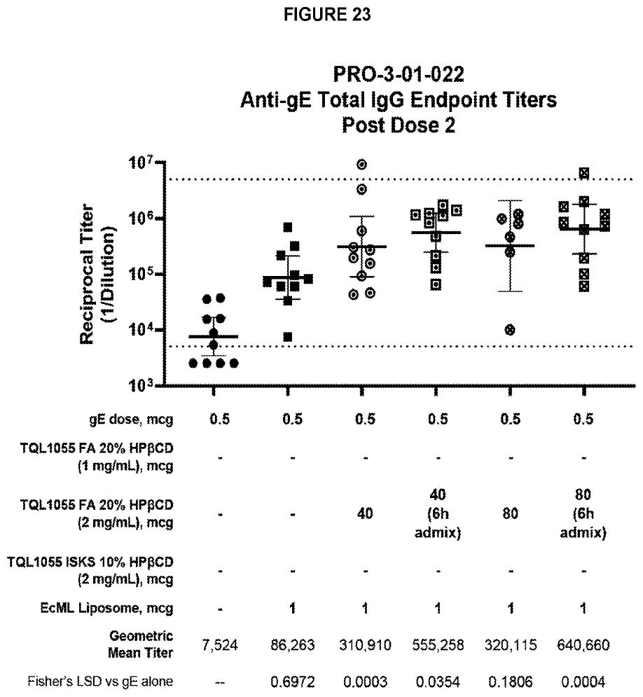

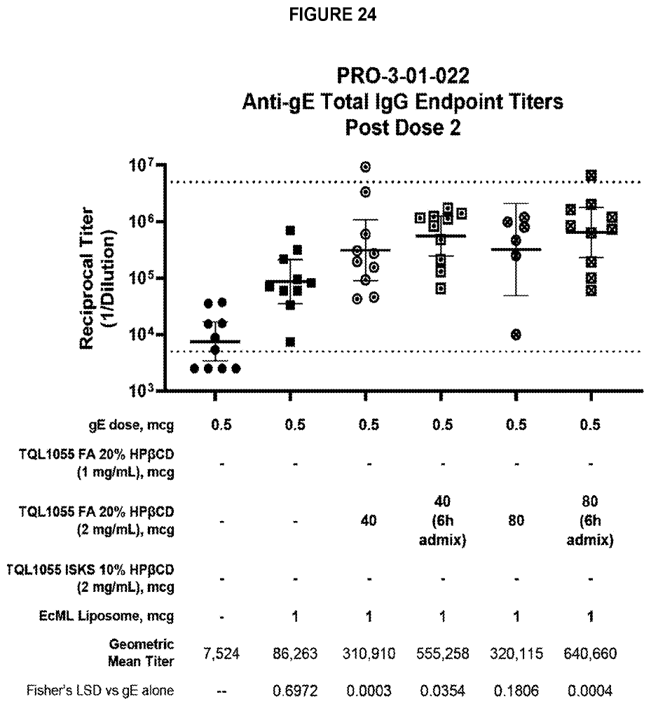

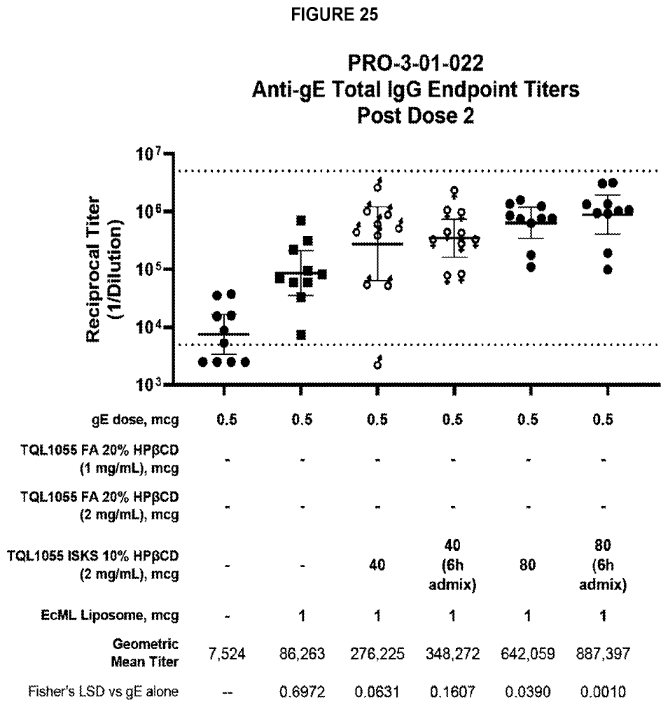

FIGS. 19 to 25 depict anti-gE total IgG endpoint titers post dose 2. Unpaired T—test P-values comparing these results are shown below.

| Unpaired T-test - 15 μg doses | | |
|---|---|---|
| Group 1 | Group 2 | P-value |
| 0.5 μg gE + 15 μg | 0.5 μg gE + 15 μg | 0.1173 |
| TQL1055 FA | TQL1055 FA | |
| 20% HPβCD (1 mg/mL) | 20% HPβCD (2 mg/mL) | |
| 0.5 μg gE + 15 μg | 0.5 μg gE + 15 μg | 0.6422 |
| TQL1055 FA | TQL1055 ISKS | |
| 20% HPβCD (1 mg/mL) | 10% HPβCD (2 mg/mL) | |
| 0.5 μg gE + 15 μg | 0.5 μg gE + 15 μg | 0.1478 |
| TQL1055 FA | TQL1055 ISKS | |
| 20% HPβCD (2 mg/mL) | 10% HPβCD (2 mg/mL) | |

| Unpaired T-test - 15 μg doses | | |
|---|---|---|
| Group 1 | Group 2 | P-value |
| 0.5 μg gE + 1 μg | 0.5 μg gE + 1 μg | 0.9399 |
| EcML + 15 μg TQL1055 | EcML + 15 μg TQL1055 | |
| FA 20% HPβCD (1 mg/mL) | FA 20% HPβCD (2 mg/mL) | |
| 0.5 μg gE + 1 μg | 0.5 μg gE + 1 μg | 0.6578 |
| EcML + 15 μg TQL1055 | EcML + 15 μg TQL1055 | |
| FA 20% HPβCD (1 mg/mL) | ISKS 10% HPβCD (2 mg/mL) | |
| 0.5 μg gE + 1 μg | 0.5 μg gE + 1 μg | 0.7418 |
| EcML + 15 μg TQL1055 | EcML + 15 μg TQL1055 | |
| FA 20% HPβCD (2 mg/mL) | ISKS 10% HPβCD (1 mg/mL) | |

| Unpaired T-test - 40 μg doses | | |
|---|---|---|
| Group 1 | Group 2 | P-value |
| 0.5 μg gE + 40 μg | 0.5 μg gE + 40 μg | 0.0068 |
| TQL1055 FA | TQL1055 FA 20% | |
| 20% HPβCD (1 mg/mL) | HPβCD (2 mg/mL) | |
| 0.5 μg gE + 40 μg | 0.5 μg gE + 40 μg | 0.0829 |
| TQL1055 FA | TQL1055 ISKS | |
| 20% HPβCD (1 mg/mL) | 10% HPβCD (2 mg/mL) | |
| 0.5 μg gE + 40 μg | 0.5 μg gE + 40 μg | 0.8433 |
| TQL1055 FA | TQL1055 ISKS | |
| 20% HPβCD (2 mg/mL) | 10% HPβCD (2 mg/mL) | |

| Unpaired T-test - 40 μg doses | | |
|---|---|---|
| Group 1 | Group 2 | P-value |
| 0.5 μg gE + 1 μg | 0.5 μg gE + 1 μg | 0.3503 |
| EcML + 40 μg TQL1055 | EcML + 40 μg TQL1055 | |
| FA 20% HPβCD (1 mg/mL) | FA 20% HPβCD (2 mg/mL) | |
| 0.5 μg gE + 1 μg | 0.5 μg gE + 1 μg | 0.5174 |
| EcML + 40 μg TQL1055 | EcML + 40 μg TQL1055 | |
| FA 20% HPβCD (1 mg/mL) | ISKS 10% HPβCD (2 mg/mL) | |
| 0.5 μg gE + 1 μg | 0.5 μg gE + 1 μg | 0.4822 |
| EcML + 40 μg TQL1055 | EcML + 40 μg TQL1055 | |
| FA 20% HPβCD (2 mg/mL) | ISKS 10% HPβCD (1 mg/mL) | |

| Unpaired T-test - 80 μg doses | | |
|---|---|---|
| Group 1 | Group 2 | P-value |
| 0.5 μg gE + 80 μg | 0.5 μg gE + 80 μg | — |
| TQL1055 FA | TQL1055 FA 20% | |
| 20% HPβCD (1 mg/mL) | HPβCD (2 mg/mL) | |
| 0.5 μg gE + 80 μg | 0.5 μg gE + 80 μg | — |
| TQL1055 FA | TQL1055 ISKS | |
| 20% HPβCD (1 mg/mL) | 10% HPβCD (2 mg/mL) | |
| 0.5 μg gE + 80 μg | 0.5 μg gE + 80 μg | 0.0313 |
| TQL1055 FA | TQL1055 ISKS | |
| 20% HPβCD (2 mg/mL) | 10% HPβCD (2 mg/mL) | |

| Unpaired T-test - 80 μg doses | | |
|---|---|---|
| Group 1 | Group 2 | P-value |
| 0.5 μg gE + 1 μg | 0.5 μg gE + 1 μg | — |
| EcML + 80 μg TQL1055 | EcML + 80 μg TQL1055 | |
| FA 20% HPβCD (1 mg/mL) | FA 20% HPβCD (2 mg/mL) | |
| 0.5 μg gE + 1 μg | 0.5 μg gE + 1 μg | — |
| EcML + 80 μg TQL1055 | EcML + 80 μg TQL1055 | |
| FA 20% HPβCD (1 mg/mL) | ISKS 10% HPβCD (2 mg/mL) | |
| 0.5 μg gE + 1 μg | 0.5 μg gE + 1 μg | 0.4061 |
| EcML + 80 μg TQL1055 | EcML + 80 μg TQL1055 | |
| FA 20% HPβCD (2 mg/mL) | ISKS 10% HPβCD (1 mg/mL) | |

| Unpaired T-test - 1 mg/ml FA [admix] | | |
|---|---|---|
| Group 1 | Group 2 | P-value |
| 0.5 μg gE + 1 μg | 0.5 μg gE + 1 μg | 0.4422 |
| EcML + 40 μg TQL1055 | EcML + 40 μg TQL1055 | |
| FA 20% HPβCD (1 mg/mL) | FA 20% HPβCD (1 mg/mL) | |
| | [6 h admix] | |

| Unpaired T-test - 2 mg/ml FA [admix] | | |
|---|---|---|
| Group 1 | Group 2 | P-value |
| 0.5 μg gE + 1 μg | 0.5 μg gE + 1 μg | 0.5360 |
| EcML + 40 μg TQL1055 | EcML + 40 μg TQL1055 | |
| FA 20% HPβCD (2 mg/mL) | FA 20% HPβCD (2 mg/mL) | |
| | [6 h admix] | |
| 0.5 μg gE + 1 μg | 0.5 μg gE + 1 μg | 0.3529 |
| EcML + 80 μg TQL1055 | EcML + 80 μg TQL1055 | |
| FA 20% HPβCD (2 mg/mL) | FA 20% HPβCD (2 mg/mL) | |
| | [6 h admix] | |

| Unpaired T-test - ISKS [admix] | | |
|---|---|---|
| Group 1 | Group 2 | P-value |
| 0.5 μg gE + 1 μg | 0.5 μg gE + 1 μg | 0.5954 |
| EcML + 40 μg TQL1055 | EcML + 40 μg TQL1055 | |
| ISKS 10% HPβCD | ISKS 10% HPβCD (2 mg/mL) | |
| (2 mg/mL) | [6 h admix] | |
| 0.5 μg gE + 1 μg | 0.5 μg gE + 1 μg | 0.1879 |
| EcML + 80 μg TQL1055 | EcML + 80 μg TQL1055 | |
| ISKS 10% HPβCD | ISKS 10% HPβCD (2 mg/mL) | |
| (2 mg/mL) | [6 h admix] | |

When used alone TQL1055 FA 20% HPβCD (1 mg/mL) tended to induce antibody responses that lagged behind TQL1055 FA 20% HPβCD (2 mg/mL) and TQL1 055 ISKS 10% HP-β-CD (2 mg/mL). When used in combination with 1 μg EcML, all TQL1055 formulations performed similarly. 6 hour admix groups induced antibody titers that trended toward increases compared to the 0 hour admix groups, but these differences did not reach statistical significance.

1. A pharmaceutical formulation comprising:
a cyclodextrin; and
a pharmaceutically acceptable salt of compound of Formula I 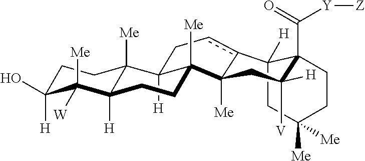
wherein [symbol]
is a single or double bond;
W is CHO;
V is hydrogen or ORx;
Y is CH₂, —O—, NR—, or NH;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure: 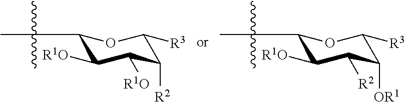
wherein each occurrence of R1 is Rx or a carbohydrate domain having the structure: 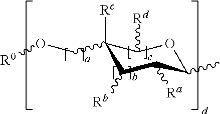
wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R0 is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C1-10 aliphatic, C1-6 heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, Rb, Rc, and Rd is independently hydrogen, halogen, OH, OR, ORx, NR2, NHCOR, or an optionally substituted group selected from acyl, C1-10 aliphatic, C1-6 heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R2 is hydrogen, halogen, OH, OR, OC(O)R4, OC(O)OR4, OC(O)NHR4, OC(O)NRR4, OC(O)SR4, NHC(O)R4, NRC(O)R4, NHC(O)OR4, NHC(O)NHR4, NHC(O)NRR4, NHR4, N(R4)₂, NHR4, NRR4, N3, or an optionally substituted group selected from C1-10 aliphatic, C1-6 heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R3 is hydrogen, halogen, CH2OR1, or an optionally substituted group selected from the group consisting of acyl, C1-10 aliphatic, C1-6 heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur, R4 is —T—Rz, —C(O)—T—Rz, —NH—T—Rz, —O—T—Rz, —S—T—Rz, —C(O)NH—T—Rz, C(O)O—T—Rz, C(O)S—T—Rz, C(O)NH—T—O—T—Rz, —O—T—Rz, —T—O—T—Rz, —T—S—T—Rz, or 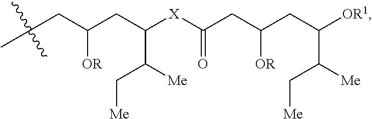
wherein
X is —O—, —NR—, or T—Rz;
T is a covalent bond or a bivalent C₁₋₂₆ saturated or unsaturated, straight or branched, aliphatic or heteroaliphatic chain; and
Rz is hydrogen, halogen, —OR, —ORx, —OR1, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of Rx is independently hydrogen or an oxygen protecting group selected from the group consisting of alkyl ethers, benzyl ethers, silyl ethers, acetals, ketals, esters, carbamates, and carbonates;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
two R on the same nitrogen atom are taken with the nitrogen atom to form a 4-7-membered heterocyclic ring having 1-2 heteroatoms independently selected from the group consisting of nitrogen, oxygen, and sulfur.

2. The pharmaceutical formulation of claim 1,
wherein the compound of Formula I is Compound I-4:

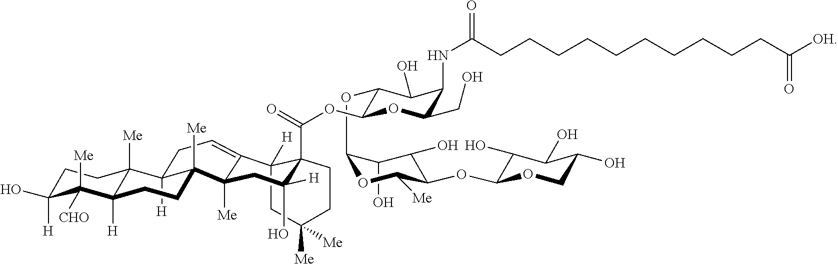

3. The pharmaceutical formulation of claim 1,
wherein the cyclodextrin is selected from the group consisting of α-cyclodextrins (αCD), β-cyclodextrins (βCD), and γ-cyclodextrin (γCD).

4. The pharmaceutical formulation of claim 1,
wherein the cyclodextrin is a β-cyclodextrin (βCD).

5. The pharmaceutical formulation of claim 1,
wherein the cyclodextrin is selected from the group consisting of HP-β-CD (2-hydroxypropyl beta-cyclodextrin) and SBECD (sulfobutylether-β-cyclodextrin).

6. The pharmaceutical formulation of claim 1,
wherein the cyclodextrin is HP-β-CD (2-hydroxypropyl beta-cyclodextrin).

7. The pharmaceutical formulation of claim 1,
wherein the cyclodextrin is of formula 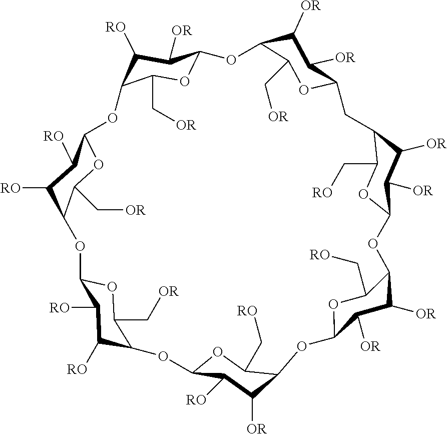
wherein each instance of R is independently H, —(CH₂)ₙ—CH₃ with n from 0 to 6, —(CH₂)ₙ—OH with n from 1 to 6, —(CH₂)ₙ—CHOH—CH₃ with n from 1 to 6, —(CH₂)ₙSO₃—Na⁺ with n from 1 to 6, glycosyl, or maltosyl.

8. The pharmaceutical formulation of claim 1,
wherein the pharmaceutically acceptable salt is a potassium salt.

9. The pharmaceutical formulation of claim 1,
further comprising an antigen.

10. The pharmaceutical formulation of claim 9,
wherein the antigen is associated with a bacteria or virus.

11. The pharmaceutical formulation of claim 10,
wherein the antigen is associated with SARS-CoV-2 virus.

12. The pharmaceutical formulation of claim 10,
wherein the antigen is associated with Varicella Zoster.

13. The pharmaceutical formulation of claim 1,
wherein the pharmaceutical formulation is stable at physiological pH for at least 12 months.

14. The pharmaceutical formulation of claim 1,
wherein the pharmaceutical formulation is stable at physiological pH for at least 24 months.

15. The pharmaceutical formulation of claim 1,
wherein the pharmaceutical formulation is stable at physiological pH for at least 36 months.

16. The pharmaceutical formulation of claim 13,
wherein physiological pH is between approximately 7.0 and 7.5.

17. The pharmaceutical formulation of claim 13,
wherein physiological pH is between approximately 7.0 and 8.0.

18. The pharmaceutical formulation of claim 13,
wherein physiological pH between approximately 7.5 and 8.0.

19. The pharmaceutical formulation of claim 13,
wherein physiological pH is approximately 7.4.

20. A method of conferring resistance to an infection, the method comprising administering an antigen in combination with a pharmaceutical formulation of claim 1.
